SEQUENCE LISTING (3 sequences)

SEQ ID NO: 1 - DNA, 26 bp, Artificial Sequence
  Synthetic forward primer
cctaagagagaagacaattggcaagt

SEQ ID NO: 2 - DNA, 35 bp, Artificial Sequence
  Synthetic reverse primer
tccatgatatctgttagtttttttcatatgtagca

SEQ ID NO: 3 - DNA, 20 bp, Artificial Sequence
  Synthetic FAM dye probe
acaaatgaccctataattct

(19) United States
(12) Patent Application Publication
(10) Pub. No.: US 2012/0171246 A1
(43) Pub. Date: Jul. 5, 2012

(54) IMMUNIZATION TO REDUCE NEUROTOXICITY DURING TREATMENT WITH CYTOLYTIC VIRUSES

(75) Inventor: Anthony N. van den Pol, Branford, CT (US)

(73) Assignee: Yale University, New Haven, CT (US)

(21) Appl. No.: 13/395,399

(22) PCT Filed: Sep. 10, 2010
(86) PCT No.: PCT/US10/48472

§ 371 Date: Mar. 9, 2012

Related U.S. Application Data
(60) Provisional application No. 61/241,092, filed on Sep. 10, 2009.

(51) Int. Cl. A61K 39/205; A61P 35/00; C12N 7/00; A61K 39/12

(52) U.S. Cl. 424/224.1; 424/204.1; 435/235.1

(57) ABSTRACT

Methods for protecting the brain and other tissues from virus-associated toxicity are provided. Patients are treated with a first immunizing virus prior to administration of a second, therapeutic cytolytic virus. The immunizing virus is preferably administered peripherally, and initiates an adaptive immune response that is sufficient to reduce or prevent the cytovirulence and inflammation caused by the therapeutic virus. The immunizing virus is administered one or more times, preferably at least twice, before administration of the therapeutic virus. The therapeutic virus can be the same virus as the immunizing virus, or a mutant or variant thereof. Preferably the therapeutic virus is an attenuated virus with reduced virulence compared to the immunizing virus.

### CROSS REFERENCE TO RELATED APPLICATIONS

This application claims priority to and benefit of U.S. Provisional Patent Application No. 61/241,092 filed on Sep. 10, 2009, and where permissible is incorporated by reference in its entirety.

### GOVERNMENT SUPPORT

The United States government has certain rights in this invention by virtue of National Institutes of Health Grant Numbers NIH A1/NS48854 and CA124737 to Anthony N. van den Pol.

### FIELD OF THE INVENTION

The present application is generally related to compositions and methods for protection against infections of normal tissue by oncolytic viruses, and attenuated oncolytic viruses for therapeutic and prophylactic use.

### BACKGROUND OF THE INVENTION

Infection of normal, healthy tissue, including brain tissue, presents a concern when considering the use of therapeutic viruses, such as oncolytic viruses for the treatment of disease and disorders. The brain occupies a special niche in viral immunity, and due to a number of mechanisms, viruses in the periphery generally do not enter the brain. However, the same mechanisms that give the brain a special protected status can also impede an immune response against intracerebral infection by viruses. Although many negative strand RNA viruses can be tolerated peripherally, CNS infection with viruses such as vesicular stomatis virus (“VSV”), rabies, measles, and influenza can be fatal for rodents and for humans (Jackson, Rabies, Neurol Clin. 26:717-726 (2008), Reiss, et al., Ann. N.Y. Acad. Sci. 855:751-761 (1998), Saha, et al., Virology, 198:129-137 (1994), Schneider-Schaulies, et al., J. Neurovirol. 5:613-22 (1994), Studahl, et al., J. Clin. Virol. 28:28:225-232 (2003)). Peripheral immunization does protect the brain from virus infections, but in most studies does so by eliminating viruses before they penetrate the blood brain barrier and get into the brain (Balachandran, J. Virol., 75:3474-79 (2001), Reuter, et al., J. Virol., 78:1473-1487 (2004), Sabin, et al., J. Exp. Med., 66:15-34 (1937), Wege, et al., Frog. Brain Res. 59:221-231 (1983)).

One replication competent virus with oncolytic potential is VSV (Lun, et al. J. Nat. Can. Instit., 98(21): 1546-1557 (2006), Wu, et al., Clin. Cancer Res., 14(4):1218-1227 (2008), Ozduman, et al., J. Virol., 83(22):11540-11549 (2009), Wollmann, et al., J. Virol, 84(3):1563-73 (2010) (epub 2010). VSV is an enveloped negative-strand RNA virus and its 11.2 kb genome codes for five viral proteins (N, P, M, G., and L). VSV is a non-human pathogen that can cause a typically self-limiting disease in livestock with flu-like symptoms (Lyles, Rhabdoviridae, p. 1363-1408, in Fields of Virology, 5^{th} ed., Linnincott Williams and Wilkins, Philadelphia, Pa. (2007).

Limiting factors of VSV for clinical use are its neurotropic properties and the still little understood potential of the brain to fight off a potential infection (Christian, et al., Virol Immuno., 9:195-205 (1996), Bi, et al., J. Virol., 69:6466-72 (1995), Johnson, et al., Virology, 360:36-49 (2007)). The brain is largely protected from viral entry through the blood-brain barrier. Mice do not show signs of CNS infection after peripheral VSV application. VSV can enter the brain directly, such as through direct intracranial injection. VSV can also spread from the periphery along a cranial nerve, for instance the olfactory nerve after intranasal administration, and can subsequently spread from the olfactory system to other regions of the brain (Reiss, et al., Ann. N.Y. Acad. Sci. 855:751-761 (1998), van den Poi, et al., J. Virol. 76:1309-1327 (2002)). In contrast to peripheral application, VSV with direct access to the CNS either experimentally through direct injection or through the intranasal path can spread through the brain, resulting in permanent neurological dysfunction in rodents or primates (Lun, et al., J. Natl. Cancer Inst., 98:1546-1557 (2006), Saha, et al., Virology, 198:129-137 (1994)) or lethal encephalitis (Flanagan, J. Virol. 77:5740-48 (2003), Johnson, et al., Virology, 360:36-49 (2007)). The replication cycle of VSV occurs in 4 to 6 hours. The rapid generation of viral progeny contributes to the quick spread of VSV in the brain. VSV spread through the brain after intranasal application is age-dependent, with mature mice showing little or no spread beyond the olfactory nerve compared to young mice succumbing to widespread viral infection throughout the brain (Lun, et al., J. Natl. Cancer Inst., 98:1546-1557 (2006), van den Pal, et al., J. Virol. 76:1309-1327 (2002)).

VSV infection triggers a fast and effective upregulation of interferon-inducible genes in the periphery followed by an induction of both the cellular and humoral branch of the systemic immune system. Outside the brain, VSV is effectively controlled and eliminated by both the innate and systemic branches of the immune system. Peripheral VSV exposure generates a strong B and T cell response that effectively controls peripheral re-challenges of the same virus. However, it is unknown to what extent a peripheral immunization can provide protection from intracranial VSV challenge.

Recombinant VSVs have shown promise in two respects: VSV can serve as a robust vaccine vector (Roberts, et al., J. Virol. 73:3723-3732 (1999), Rose, et al., Cell, 106:539-49 (2001), Jones, et al., Nat. Med. 11:786-790 (2005)) and as a potent oncolytic virus against a variety of peripheral (Ahmed, et al., Virology, 330:34-49 (2004), Balachandran, et al., J. Virol. 197:669-677 (1993), Ebert, et al., Cancer Gene Ther., 12:350-358 (2005), Stojdl, et al., Cancer Cell, 4:263-275 (2003)) or central nervous system tumors (Duntsch, et al., J. Neurosurg., 100:1049-59 (2004), Lundh, et al., J. Neuropathol. Exp. Neural. 47:497-506 (1988), Ozduman, et al., J. Neurosci., 28:1882-1893 (2008), Wollman, et al., J. Virol. 79:6005-6022 (2005), Wollmann, et al., J. Virol. 81:1479-1491 (2007)). A number of studies have shown the protective effects of peripheral immunization with VSV on peripheral viral infections (Gearhart, et al., J. Am. Vet. Med. Assoc., 191:819-822 (1987), Gobet, et al., Exp. Cell Bio., 56:175-180 (1988)). The effect of peripheral immunization on viral infections within the brain has received considerably less attention. The extent of VSV pathogenesis in the brain is determined by the replicative efficacy of the virus and the efficiency of the host immune response to curb the infection. Modification of either of these components can alter the course and extent of CNS damage. Little is known about to what extent the adaptive immune response can influence VSV within the brain. Both as a vaccine vector and as an oncolytic virus, VSV infection of normal brain cells remains a concern.

Therefore, it is an objection of the invention to provide compositions including immunogenic viruses and methods of using them to reduce the neurovirulence of therapeutic viruses.

It is another object of the invention to provide an oncolytic vesicular stomatitis virus having both an M51 deletion and G protein truncation.

It is also an object of the invention to provide methods of treating cancer using therapeutic viruses with reduced neurovirulence.

It is also an object of the invention to provide methods of vaccination using VSV vectors with reduced neurovirulence.

It is a further object of the invention to provide a method of treating cancer with an attenuated oncolytic vesicular stomatitis virus having both an M51 deletion and G protein truncation.

### SUMMARY OF THE INVENTION

It has been discovered that peripheral immunization with oncolytic virus blocks the lethal consequences of oncolytic virus injected directly into the brain and provides complete protection from both attenuated and non-attenuated oncolytic viruses. Immunization also prevents the neurological dysfunction associated with CNS infections of VSV. Peripheral innoculation, also referred to herein as prime vaccination or immunization, with a first immunizing virus prior to administration of a second, therapeutic virus reduces the neuroinvasiveness and/or neurovirulence of the therapeutic virus. Immunizing and therapeutic cytolytic viruses are typically administered to a patient in need thereof in a pharmaceutical composition. The immunizing virus initiates an adaptive immune response that is sufficient to reduce or prevent the cytotoxicity and inflammation in the normal tissues caused by the therapeutic virus. The immunizing virus is administered one or more times, preferably at least twice, before administration of the therapeutic virus. Preferably the immunizing virus is administered intranasally or by intramuscular injection. The subject's adaptive immune response can be monitored to assess the effectiveness of the immunization.

The therapeutic virus can be the same virus as the immunizing virus, or a mutant or variant thereof. Preferably the therapeutic virus is an attenuated virus with reduced virulence compared to the immunizing virus. In the most preferred embodiment, therapeutic virus is delivered locally, for example, by intracranial injection. Viruses may be modified to express one or more targeting or therapeutic proteins, separately or as a part of other expressed proteins. In some embodiments the therapeutic virus is an oncolytic virus used in the treatment of a tumor. In some embodiments the therapeutic virus is a vaccine vector expressing an immunogenic antigen. The immunogenic antigen produces prophylactic or therapeutic immunity against a diseases or disorder, when expressed by the VSV virus administered to a patient in need thereof.

The disclosed methods can also be coupled with surgical, radiologic, other therapeutic approaches to treatment of tumors, and in combination with other known pharmaceutical and/or pharmacological therapies. In some cases it may be desirable to immunosuppress the individual after administration of the vaccine and cytolytic virus, to insure that the cytolytic virus is able to proliferate and kill the targeted tissue, such as one or more tumors, without inhibition by the immune system primed by the vaccine.

### BRIEF DESCRIPTION OF THE DRAWINGS

FIG. 1A is a line graph showing the percent (%) survival of 16-day-old mice over time (days post inoculation (DPI)) following administration of 1,500 plaque forming units (PFU) of VSV-0/GFP (n=7), or VSV-CT1 (n=7), or VSV-CT9-M51 (n=7) by intracerebral injection. FIG. 1B is a line graph showing the percent (%) survival of 16-day-old mice over time (days post inoculation (DPI)) following administration of 10,000 plaque forming units (PFU) of VSV-G/GFP (n=8), or VSV-CT1 (n=9), or VSV-CT9-M51 (n=8) by intranasal inoculation. FIG. 1C is a line graph showing the percent (%) survival of adult mice over time (days postinoculation (DPI)) following administration of 15,000 plaque forming units (PFU) of VSV-G/GFP (n=10), or VSV-CT1 (n=7), or VSV-CT9-M51 (n=19) by intracerebral injection.

FIG. 2A is a bar graph showing the relative number of copies of viral genome (%) detected by quantitative PCR of a region of the N gene of VSV in samples of reverse-transcribed RNA from homogenized brains of mice injected with VSV-G/GFP or VSV-CT9-M51. Error bars indicated standard error of the mean, FIG. 2B is a bar graph of an EthD-1 plaque assay, showing the relative sizes of plaques (VSV plaque diameter (μm)) in cultures of normal human mouse brain infected with 1001 FU of VSV-G/GFP or VSV-CT9-M51.

FIGS. 3A, 3B, and 3C are line graphs showing the percent (%) survival of five-week-old mice with (solid lines) or without (hatched lines) peripheral immunization as a function of time (days postinoculation (DPI)) after administration of VSV-G/GFP (FIG. 3A), or VSV-CT1 (FIG. 3B), or VSV-CT9-M51 (FIG. 3B) by intracerebral injection. Error bars indicated standard errors.

FIGS. 4A, 4B, and 4C are line graphs showing the percent (%) body weight of five-week-old mice with (--) or without (-□-) peripheral immunization as a function of time (days postinoculation (DPI)) after administration of VSV-G/GFP (FIG. 3A), or VSV-CT1 (FIG. 3B), or VSV-CT9-M51 (FIG. 3B) by intracerebral injection. Error bars indicated standard errors.

FIGS. 5A and 5B are line graphs showing the percent (%) body weight of immunized (--) and non-immunized (-□-) five-week-old mice after primary (FIG. 5A) and secondary (booster) (FIG. 5B) peripheral immunization as a function of time after immunization (days postinoculation (DPI)). Error bars indicated standard errors.

FIGS. 6A and 6B are bar graphs showing the expression (fold induction) of two interferon downstream genes, MxA (FIG. 6A) and ISG15 (FIG. 6B) as measured with quantitative reverse transcription (RT)-PCR in primary cultures of adult human astrocytes after 6 hours incubation with VSV-G/GFP or VSV-CT9-M51. Error bars indicated standard errors.

FIG. 7 is a bar graph of IRF3 nuclear translocation showing the % nuclear IRF3 positive cells in adult human astrocytes with no virus (control) or VSV-G/GFP infection or VSV-CT9-M51 infection at 8 (hatched bars) and 12 hours (open bars) postinoculation (HPI).

### DETAILED DESCRIPTION OF THE INVENTION

### I. Definitions

As used herein the term “isolated” describes a compound of interest (e.g., either a polynucleotide or a polypeptide) that is in an environment different from that in which the compound naturally occurs e.g. separated from its natural milieu such as by concentrating a peptide to a concentration at which it is not found in nature. “Isolated” includes compounds that are within samples that are substantially enriched for the compound of interest and/or in which the compound of interest is partially or substantially purified. With respect to nucleic acids, the term “isolated” includes any non-naturally-occurring nucleic acid sequence, since such non-naturally-occurring sequences are not found in nature and do not have immediately contiguous sequences in a naturally-occurring genome.

As used herein, the term “nucleic acid(s)” refers to any nucleic acid containing molecule, including, but not limited to, DNA or RNA. The term encompasses sequences that include any of the known base analogs of DNA and RNA including, but not limited to, 4-acetylcytosine, 8-hydroxy-N-6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxy-aminomethyl-2-thiouracil, beta-D-mannosylqueosine, 5′-methoxycarbonylmethyluracil, 5-methoxyuracil, 2-methylthio-N-6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine. In accordance with standard nomenclature, nucleic acid sequences are denominated by either a three letter, or single letter code as indicated as follows: adenine (Ade, A), thymine (Thy, T), guanine (Gua, G) cytosine (Cyt, C), uracil (Ura, U).

As used herein, the term “polynucleotide” refers to a chain of nucleotides of any length, regardless of modification (e.g., methylation).

The term “gene” refers to a nucleic acid (e.g., DNA or RNA) sequence that comprises coding sequences necessary for the production of a polypeptide, RNA (e.g., including but not limited to, mRNA, tRNA and rRNA) or precursor. The polypeptide, RNA, or precursor can be encoded by a full length coding sequence or by any portion thereof. The term also encompasses the coding region of a structural gene and the sequences located adjacent to the coding region on both the 5′ and 3′ ends for a distance of about 1 kb on either end such that the gene corresponds to the length of the full-length mRNA. The term “gene” encompasses both cDNA and genomic forms of a gene, which may be made of DNA, or RNA. A genomic form or clone of a gene may contain the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “nucleic acid molecule encoding,” refers to the order or sequence of nucleotides along a strand of nucleotides. The order of these nucleotides determines the order of amino acids along the polypeptide (protein) chain. The nucleotide sequence thus codes for the amino acid sequence

As used herein, the term “polypeptide” refers to a chain of amino acids of any length, regardless of modification (e.g., phosphorylation or glycosylation). In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (H is, H), Isoleucine (Ile, I), Leucine (Leu, L), Lysine (Lys, K), Methionine (Met, M), Phenylalanine (Ph; F), Proline (Pro, P), Serine (Ser, 5), Threonine (Thr, T), Tryptophan (Tip, W), Tyrosine (Tyr, Y), and Valine (Val, V).

As used herein, a “variant,” “mutant,” or “mutated” polynucleotide contains at least one polynucleotide sequence alteration as compared to the polynucleotide sequence of the corresponding wild-type or parent polynucleotide. Mutations may be natural, deliberate, or accidental. Mutations include substitutions, deletions, and insertions.

As used herein, a “nucleic acid sequence alteration” can be, for example, a substitution, a deletion, or an insertion of one or more nucleotides. An “amino acid sequence alteration” can be, for example, a substitution, a deletion, or an insertion of one or more amino acids.

As used herein, the term “immunizing virus” includes infectious virus, viral subunits, viral proteins and antigenic fragments thereof, nucleic acids encoding viral subunits, antigenic proteins or polypeptides, and expression vectors containing the nucleic acids.

As used herein, a “vector” is a replicon, such as a plasmid, phage, or cosmid, into which another DNA segment may be inserted so as to bring about the replication of the inserted segment. The vectors described herein can be expression vectors.

As used herein, the term “effective amount” or “therapeutically effective amount” means a dosage sufficient to treat, inhibit, or alleviate one or more symptoms of a disease state being treated or to otherwise provide a desired pharmacologic and/or physiologic effect. The precise dosage will vary according to a variety of factors such as subject-dependent variables (e.g., age, immune system health, etc.), the disease, and the treatment being effected.

As used herein, the terms “neoplastic cells,” “neoplasia,” “tumor,” “tumor cells,” “cancer” and “cancer cells,” (used interchangeably) refer to cells which exhibit relatively autonomous growth, so that they exhibit an aberrant growth phenotype characterized by a significant loss of control of cell proliferation (i.e., de-regulated cell division). Neoplastic cells can be malignant or benign.

As used herein, an immunogen or immunogenic amount refers to the ability of a substance (antigen) to induce an immune response. An immune response is an alteration in the reactivity of an organisms immune system in response to an antigen, in vertebrates, this may involve antibody production, induction of cell-mediated immunity, complement activation or development of immunological tolerance.

As used herein, an adjuvant is a substance that increases the ability of an antigen to stimulate the immune system.

As used herein, attenuated refers to refers to procedures that weaken an agent of disease (a pathogen). An attenuated virus is a weakened, less vigorous virus. A vaccine against a viral disease can be made from an attenuated, less virulent strain of the virus, a virus capable of stimulating an immune response and creating immunity but not causing illness or less severe illness. Attenuation can be achieved by chemical treatment of the pathogen, through radiation, or by genetic modification, using methods known to those skilled in the art. Attenuation may result in decreased proliferation, attachment to host cells, or decreased production or strength of toxins.

### II. Methods of Use

VSV has gained attention as a promising biological anticancer agent against a variety of malignancies. Malignant brain tumors have been successfully targeted by VSV in laboratory studies, but viral spread through the CNS and neurotoxicity remain challenges to be addressed. Methods for protecting the brain from oncolytic virus-associated neurovirulence are provided. It has been discovered that peripheral inoculation, also referred to herein as prime vaccination or immunization, with a first immunizing virus prior to administration of a second, therapeutic virus reduces the neuroinvasiveness and/or neurovirulence of the therapeutic virus. This is particularly useful when treating a patient in need thereof with a therapeutic oncolytic virus administered directly to the brain. The prime vaccination protects the brain from damage, neurological dysfunction, and potentially lethal conditions such as encephalitis that can be caused by administration of the therapeutic virus. In some embodiments the immunizing virus is the same virus as the therapeutic virus.

A. Compositions

1. Selection of Virus

The viruses used in the methods disclosed herein may be “native” or naturally-occurring viruses, or genetically engineered viruses, such as recombinant viruses. The immunizing virus may be any suitable virus. The therapeutic virus can be the same virus as the immunizing virus, or a mutant or variant thereof. Alternatively, the therapeutic virus can be a different virus, for example, a virus derived from a different parental strain or different serotype than the therapeutic virus.

The extent of pathogenesis of a therapeutic virus is determined by a number of factors including replicative efficacy of the virus and the efficiency of the host immune response in controlling the infection. Therefore, the safety and effectiveness of the disclosed methods can be enhanced by utilizing an attenuated therapeutic virus. Mutations can be introduced into the viral genome to provide viruses that have an attenuated phenotype and preferably have reduced neurovirulence in the brain when compared to wildtype virus, or the parental strain. In preferred embodiments, the therapeutic virus and the immunizing virus are VSV viruses.

a. Oncolytic Viruses

VSV has gained attention as a promising biological anticancer agent against a variety of malignancies. Malignant brain tumors have been successfully targeted by VSV in laboratory studies (Lundh, et al., J. Neurophatol. Exp. Neurol., 47:497-506 (1988) Ozduman, et al., J. Neurosci., 28:1882-1893 (2008)). Useful VSV viruses can be viruses that are known in the art, or they can be mutants or variants of known viruses. Any suitable VSV strain or serotype may be used, including, but not limited to, VSV Indiana, VSV New Jersey, VSV Chandipura, VSV Isfahan, VSV San Juan, or VSV Glasgow. Viruses can be naturally occurring viruses, or modified, for example, to increase or decrease the virulence of the virus, and/or increase the specificity or infectivity of the virus compared to the parental strain. It may be desirable to further reduce the neurovirulence the viruses used in the disclosed methods, particularly the virulence of the therapeutic virus, by using an attenuated virus. A number of suitable VSV mutants have been described, see for example (Clarke, et al., J. Virol., 81:2056-64 (2007), Flanagan, et al., J. Virol., 77:5740-5748 (2003), Johnson, et al., Virology, 360:36-49 (2007), Simon, et al., J. Virol., 81:2078-82 (2007), Stojdl, et al., Cancer Cell, 4:263-275 (2003)), Wollmann, et al., J Virol, 84(3):1563-73 (2010) (epub 2010), WO 10/080,909, U.S. Published Application No. 200710218078, and U.S. Published Application No 2009/0175906. Recombinant VSVs derived from DNA plasmids in general show weakened virulence (Rose, et al., Cell, 106:539-549 (2001)). Attenuation of VSV phenotype can also be accomplished by one or more nucleotide sequence alterations that result in substitution, deletion, or insertion of one or more amino acids of the polypeptide it encodes.

i. G Protein Mutants

It may be desirable to attenuate virus growth, and/or block the ability to produce infectious virus, (Duntsch, et al., J. Neurosurg., 100:1049-1059 (2004)), for example by deleting or mutating the viral glycoprotein (G protein). The VSV transmembrane G protein is needed for binding and internalization, and truncations in the G protein to generate a reduced number of cytoplasmic amino acids result in attenuated virus (Johnson, et al., Virology, 360:36-49 (2007), Schnell, et al., EMBO J., 17:1289-1296)). In some embodiments 1, 2, 3, 4, 5, or more amino acids are deleted from the G protein. For example the cytoplasmic portion of the G protein can be truncated from 29 amino acids to nine amino acids (VSV-CT9) or a single amino acid (VSV-CT1). VSV-CT1 and VSV-CT9 were made in Jack Rose lab for use in immunization, as described by Schnell et al. (1998) EMBO 17: 1289-1296. PMID: 9482726.

ii. M Protein Mutants

Another strategy is to attenuate viral pathogenicity by reducing the ability of the virus to suppress host innate immune responses without compromising the yield of infectious progeny. This can be accomplished by mutating the M protein as described, for example, in Ahmed, J. Virol., 82(18):9273-9277 (2008). The M protein is a multifunctional protein that is involved in the shutoff of host transcription, nuclear cytoplasmic transport, and translation during virus infection (Lyles, Microbiol. Mol. Biol. Rev. 64:709-724 (2000)). Mutation and/or deletion of one or more amino acids from the M protein, for example MΔ51, or M51 A can result in viral protein that is defective at inhibiting host gene expression. It may also be desirable to switch or combine various substitutions, deletions, and insertions to further modify the phenotype of the virus. In a preferred embodiment, the attenuated VSV has both a truncation of the cytoplasmic tail of the G protein, and a deletion or mutation in the M protein. In the most preferred embodiment, the attenuated VSV is VSV-CT9-M51, characterized by a truncation of the cytoplasmic tail of the G protein to 9 amino acids and a deletion of the fifty-first (51) amino acid of the M protein. VSV-CT9-M51 viruses may or may not contain a GFP reporter gene inserted between the G and L genes. The VSVCT9-M51 described in the examples below was constructed by Jack Rose's lab, and is described in Wollmann et al, 2010 J Virol 84: 1563-73. It is derived from a recombinant version of the San Juan strain of Indiana serotype VSV, the genome of which consists of a single negative strand of RNA that encodes five genes, N, P, M, G and L. As described in the examples below, it has been discovered that viruses having both an M protein deletion, and trunctation of the cytoplasmic tail retain oncolytic activity, yet have reduced neurovirulence.

iIi. Gene Switching and Rearrangement

Altering the order of genes can also be used to attenuate virus (Clarke, et al., J. Virol., 81:2056-2064, (2007), Cooper, et al., J. Virol., 82:207-219 (2008), Flanagan, et al., J. Virol., 75:6107-6114 (2001)). VSV is highly immunogenic, and a substantial B and T cell response from the adaptive immune system will ultimately limit VSV infection, which will halt runaway long-lasting viral infections. A virus that shows enhanced selectivity, and a faster rate of infection, will have a greater likelihood of eliminating cancer cells before the virus is eliminated by the immune system. However, the use of VSV against cancer cells does not have to be restricted to a single application. By molecular substitution of the G-protein for enhancing immune responses against foreign genes expressed by VSV, one could switch the original Indiana G protein of the virus with the G protein from VSV New Jersey or Chandipura, allowing a slightly different antigen presentation, and reducing the initial response of the adaptive immune system to second or third oncolytic inoculations with VSV.

It also may be desirable to rearrange the VSV genome. For example, shifting the L-gene to the sixth position, by rearrangement or insertion of an additional gene upstream, can result in attenuated L-protein synthesis and a slight reduction in replication (Dalton and Rose, Virology, 279(2):414-21 (2001)), an advantage when considering treatment of the brain.

iv. Adaptive Passaging

Repeat passaging of virulent strains under evolutionary pressure can also be used to generate attenuated virus, increase specificity of the virus for a particular target cells, and/or increase the oncolytic potential of the virus. For example, VSV-rp30 (“30 times repeated passaging”) is a wild-type-based VSV with an enhanced oncolytic profile (Wollmann, et al., J. Virol. 79:6005-6022 (2005)). As described in WO10/080,909, VSV-rp30 has a preference for glioblastoma over control cells and an increased cytolytic activity on brain tumor cells.

b. Viruses Engineered to Express Targeting or Therapeutic Proteins

Viruses may be modified to express one or more targeting or therapeutic proteins, separately or as a part of other expressed proteins. The viral genome of VSV has the capacity to accommodate additional genetic material. At least two additional transcription units, totaling 4.5 kb, can be added to the genome, and methods for doing so are known in the art. The added genes are stably maintained in the gnome upon repeated passage (Schnell, et al., EMBO Journal, 17:1289-1296 (1998); Schnell, et al., PNAS, 93: 11359-11365 (1996); Schnell, et al., Journal of Virology, 70:2318-2323 (1996); Kahn, et al., Virology, 254, 81-91 (1999)).

Viruses can be engineered to include one or more additional genes that target the virus to cells of interest, see for example U.S. Pat. No. 7,429,481. In preferred embodiments, expression of the gene results in expression of a ligand on the surface of the virus containing one or more domains that bind to antigens, ligands or receptors that are specific to tumor cells, or are upregulated in tumor cells compared to normal tissue. Appropriate targeting ligands will depend on the cell or cancer of interest and will be known to those skilled in the art.

For example, virus can be engineered to bind to antigens or receptors that are specific to tumor cells or tumor-associated neovasculature, or are upregulated in tumor cells or tumor-associated neovasculature compared to normal tissue.

i. Antigens, Ligands, and Receptors to Target

(1) Tumor-Specific and Tumor-Associated Antigens

In one embodiment the viral surface contains a domain that specifically binds to an antigen that is expressed by tumor cells. The antigen expressed by the tumor may be specific to the tumor, or may be expressed at a higher level on the tumor cells as compared to non-tumor cells. Antigenic markers such as serologically defined markers known as tumor associated antigens, which are either uniquely expressed by cancer cells or are present at markedly higher levels (e.g., elevated in a statistically significant manner) in subjects having a malignant condition relative to appropriate controls, are known.

Tumor-associated antigens may include, for example, cellular oncogene-encoded products or aberrantly expressed proto-oncogene-encoded products (e.g., products encoded by the neu, ras, trk, and kit genes), or mutated forms of growth factor receptor or receptor-like cell surface molecules (e.g., surface receptor encoded by the c-erb B gene). Other tumor-associated antigens include molecules that may be directly involved in transformation events, or molecules that may not be directly involved in oncogenic transformation events but are expressed by tumor cells (e.g., carcinoembryonic antigen, CA-125, melanoma associated antigens, etc.) (see, e.g., U.S. Pat. No. 6,699,475; Jager, et al., Int. J. Cancer, 106:817-20 (2003); Kennedy, et al., Int. Rev. Immunol., 22:141-72 (2003); Scanlan, et al. Cancer Immun., 4:1 (2004)).

Genes that encode cellular tumor associated antigens include cellular oncogenes and proto-oncogenes that are aberrantly expressed. In general, cellular oncogenes encode products that are directly relevant to the transformation of the cell, so these antigens are particularly preferred targets for oncotherapy and immunotherapy. An example is the tumorigenic neu gene that encodes a cell surface molecule involved in oncogenic transformation. Other examples include the ras, kit, and trk genes. The products of proto-oncogenes (the normal genes which are mutated to form oncogenes) may be aberrantly expressed (e.g., overexpressed), and this aberrant expression can be related to cellular transformation. Thus, the product encoded by proto-oncogenes can be targeted. Some oncogenes encode growth factor receptor molecules or growth factor receptor-like molecules that are expressed on the tumor cell surface. An example is the cell surface receptor encoded by the c-erbB gene. Other tumor-associated antigens may or may not be directly involved in malignant transformation. These antigens, however, are expressed by certain tumor cells and may therefore provide effective targets. Some examples are carcinoembryonic antigen (CEA), CA 125 (associated with ovarian carcinoma), and melanoma specific antigens.

In ovarian and other carcinomas, for example, tumor associated antigens are detectable in samples of readily obtained biological fluids such as serum or mucosal secretions. One such marker is CAl25, a carcinoma associated antigen that is also shed into the bloodstream, where it is detectable in serum (e.g., Bast, et al., N Eng. J. Med., 309:883 (1983); Lloyd, et al., Int. J. Canc., 71:842 (1997). CAl25 levels in serum and other biological fluids have been measured along with levels of other markers, for example, carcinoembryonic antigen (CEA), squamous cell carcinoma antigen (SCC), tissue polypeptide specific antigen (TPS), sialyl TN mucin (STN), and placental alkaline phosphatase (PLAP), in efforts to provide diagnostic and/or prognostic profiles of ovarian and other carcinomas (e.g., Sarandakou, et al., Acta Oncol., 36:755 (1997); Sarandakou, et al., Eur. J. Gynaecol. Oncol., 19:73 (1998); Meier, et al., Anticancer Res., 17(4B):2945 (1997); Kudoh, et al., Gynecol. Obstet. Invest., 47:52 (1999)). Elevated serum CAl25 may also accompany neuroblastoma (e.g., Hirokawa, et al., Surg. Today, 28:349 (1998), while elevated CEA and SCC, among others, may accompany colorectal cancer (Gebauer, et al., Anticancer Res., 17(4B):2939 (1997)).

The tumor associated antigen mesothelin, defined by reactivity with monoclonal antibody K-1, is present on a majority of squamous cell carcinomas including epithelial ovarian, cervical, and esophageal tumors, and on mesotheliomas (Chang, et al., Cancer Res., 52:181 (1992); Chang, et al., Int. J. Cancer, 50:373 (1992); Chang, et al., Int. J. Cancer, 51:548 (1992); Chang, et al., Proc. Natl. Acad. Sci. USA, 93:136 (1996); Chowdhury, et al., Proc. Natl. Acad. Sci. USA, 95:669 (1998)). Using MAb K-1, mesothelin is detectable only as a cell-associated tumor marker and has not been found in soluble form in serum from ovarian cancer patients, or in medium conditioned by OVCAR-3 cells (Chang, et al., Int. J. Cancer, 50:373 (1992)). Structurally related human mesothelin polypeptides, however, also include tumor-associated antigen polypeptides such as the distinct mesothelin related antigen (MRA) polypeptide, which is detectable as a naturally occurring soluble antigen in biological fluids from patients having malignancies (see WO 00/50900).

A tumor antigen may include a cell surface molecule. Tumor antigens of known structure and having a known or described function, include the following cell surface receptors: HER1 (GenBank Accession NO: U48722), HER2 (Yoshino, et al., J. Immunol., 152:2393 (1994); Disis, et al., Cane. Res., 54:16 (1994); GenBank Ace. Nos. X03363 and M17730), HER3 (GenBank Ace. Nos. U29339 and M34309), HER4 (Plowman, et al., Nature, 366:473 (1993); GenBank Ace. Nos. L07868 and T64105), epidermal growth factor receptor (EGFR) (GenBank Ace. Nos. U48722, and KO3193), vascular endothelial cell growth factor (GenBank NO: M32977), vascular endothelial cell growth factor receptor (GenBank Acc. Nos. AF022375, 1680143, U48801 and X62568), insulin-like growth factor-1 (GenBank Ace. Nos. X00173, X56774, X56773, X06043, European Patent No. GB 2241703), insulin-like growth factor-II (GenBank Acc. Nos. X03562, X00910, M17863 and M17862), transferrin receptor (Trowbridge and Omary, Proc. Nat. Acad. USA, 78:3039 (1981); GenBank Ace. Nos. X01060 and M11507), estrogen receptor (GenBank Ace. Nos. M38651, X03635, X99101, U47678 and M12674), progesterone receptor (GenBank Ace. Nos. X51730, X69068 and M15716), follicle stimulating hormone receptor (FSH-R) (GenBank Acc. Nos. 234260 and M65085), retinoic acid receptor (GenBank Ace. Nos. L12060, M60909, X77664, X57280, X07282 and X06538), MUC-1 (Barnes, et al., Proc. Nat. Acad. Sci. USA, 86:7159 (1989); GenBank Ace. Nos. M65132 and M64928) NY-ESO-1 (GenBank Ace. Nos. AJ003149 and U87459), NA 17-A (PCT Publication NO: WO 96/40039), Melan-A/MART-1 (Kawakami, et al., Proc. Nat. Acad. Sci. USA, 91:3515 (1994); GenBank Ace. Nos. U06654 and U06452), tyrosinase (Topalian, et al., Proc. Nat. Acad. Sci. USA, 91:9461 (1994); GenBank Ace. NO: M26729; Weber, et al., J. Clin. Invest, 102:1258 (1998)), Gp-100 (Kawakami, et al., Proc. Nat. Acad. Sci. USA, 91:3515 (1994); GenBank Ace. NO: 573003, Adema, et al., J Biol. Chem., 269:20126 (1994)), MAGE (van den Bruggen, et al., Science, 254:1643 (1991)); GenBank Ace. Nos. U93163, AF064589, U66083, D32077, D32076, D32075, U10694, U10693, U10691, U10690, U10689, U10688, U10687, U10686, U10685, L18877, U10340, U10339, L18920, U03735 and M77481), BAGE (GenBank Ace. NO: U19180; U.S. Pat. Nos. 5,683,886 and 5,571,711), GAGE (GenBank Acc. Nos. AF055475, AF055474, AF055473, U19147, U19146, U19145, U19144, U19143 and U19142), any of the CTA class of receptors including in particular HOMMEL-40 antigen encoded by the SSX2 gene (GenBank Acc. Nos. X86175, U90842, U90841 and X86174), carcinoembryonic antigen (CEA, Gold and Freedman, J. Exp. Med., 121:439 (1985); GenBank Ace. Nos. M59710, M59255 and M29540), and PyLT (GenBank Acc. Nos. J02289 and J02038); p97 (melanotransferrin) (Brown, et al., J. Immunol., 127:539-46 (1981); Rose, et al., Proc. Natl. Acad. Sci, USA, 83:1261-61 (1986)).

Additional tumor associated antigens include prostate surface antigen (PSA) (U.S. Pat. Nos. 6,677,157; 6,673,545); n-human chorionic gonadotropin β-HCG) (McManus, et al., Cancer Res., 36:3476-81 (1976); Yoshimura, et al., Cancer, 73:2745-52 (1994); Yamaguchi, et al., Br. J. Cancer, 60:382-84 (1989): Alfthan, et al., Cancer Res., 52:4628-33 (1992)); Glycosyltransferase β-1,4-N-acetylgalactosaminyltransferases (GalNAc) (Hoon, et al., Int. J. Cancer, 43:857-62 (1989); Ando, et al., Int. J. Cancer, 40:12-17 (1987); Tsuchida, et al., J. Natl. Cancer, 78:45-54 (1987); Tsuchida, et al., J. Natl. Cancer, 78:55-60 (1987)); NUC18 (Lehmann, et al., Proc. Natl. Acad. Sci. USA, 86:9891-95 (1989); Lehmann, et al., Cancer Res., 47:841-45 (1987)); melanoma antigen gp75 (Vijayasardahi, et al., J. Exp. Med., 171:1375-80 (1990); GenBank Accession NO: X51455); human cytokeratin 8; high molecular weight melanoma antigen (Natali, et al., Cancer, 59:55-63 (1987); keratin 19 (Datta, et al., J. Clin. Oncol., 12:475-82 (1994)).

Tumor antigens of interest include antigens regarded in the art as “cancer/testis” (CT) antigens that are immunogenic in subjects having a malignant condition (Scanlan, et al., Cancer Immun., 4:1 (2004)). CT antigens include at least 19 different families of antigens that contain one or more members and that are capable of inducing an immune response, including, but not limited to, MAGEA (CT1); BAGE (CT2); MAGEB (CT3); GAGE (CT4); SSX (CT5); NY-ESO-1 (CT6); MAGEC(CT7); SYCP1 (C8); SPANXB1 (CT11.2); NA88 (CT18); CTAGE (CT21); SPA17 (CT22); OY-TES-1 (CT23); CAGE (CT26); HOM-TES-85 (CT28); HCA661 (CT30); NY-SAR-35 (CT38); FATE (CT43); and TPTE (CT44).

Additional tumor antigens that can be targeted, including a tumor-associated or tumor-specific antigen, include, but are not limited to, alpha-actinin-4, Ber-Abl fusion protein, Casp-8, beta-catenin, cdc27, cdk4, cdkn2a, coa-1, dek-can fusion protein, EF2, ETV6-AML1 fusion protein, LDLR-fucosyltransferaseAS fusion protein, HLA-A2, HLA-A11, hsp70-2, KIAAO205, Mart2, Mum-1, 2, and 3, neo-PAP, myosin class I, OS-9, pml-RARα fusion protein, PTPRK, K-ras, N-ras, Triosephosphate isomeras, Bage-1, Gage 3,4,5,6,7, GnTV, Herv-K-mel, Lage-1, Mage-A1,2,3,4,6,10,12, Mage-C2, NA-88, NY-Eso-1/Lage-2, SP17, SSX-2, and TRP2-Int2, MelanA (MART-1), gp100 (Pmel 17), tyrosinase, TRP-1, TRP-2, MAGE-1, MAGE-3, BAGE, GAGE-1, GAGE-2, p15(58), CEA, RAGE, NY-ESO (LAGE), SCP-1, Hom/MeI-40, PRAME, p53, H-Ras, HER-2/neu, BCR-ABL, E2A-PRL, 114-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, p180erbB-3, c-met, nm-PSA, TAG-72-4, CA 19-9, CA 72-4, CAM 17.1, NuMa, K-ras, β-Catenin, CDK4, Mum-1, p16, TAGE, PSMA, PSCA, CT7, telomerase, 43-9F, 5T4, 791Tgp72, α-fetoprotein, 13HCG, BCA225, BTAA, CA 125, CA 15-3 (CA 27.29\BCAA), CA 195, CA 242, CA-50, CAM43, CD681 KP1, CO-029, FGF-5, G250, Ga733 (EpCAM), HTgp-175, M344, MA-50, MG7-Ag, MOV18, NB\70K, NY—CO-1, RCAS1, SDCCAG16, TA-90 (Mac-2 binding protein\cyclophilin C-associated protein), TAAL6, TAG72, TLP, and TPS. Other tumor-associated and tumor-specific antigens are known to those of skill in the art and are suitable for targeting by the disclosed fusion viruses.

(2) Antigens Associated with Tumor Neovasculature

Oncolytic viral therapeutics can be more effective in treating tumors by targeting to blood vessels of the tumor. Tumor-associated neovasculature provides a readily accessible route through which viral therapeutics can access the tumor. In one embodiment the viral proteins contain a domain that specifically binds to an antigen that is expressed by neovasculature associated with a tumor.

The antigen may be specific to tumor neovasculature or may be expressed at a higher level in tumor neovasculature when compared to normal vasculature. Exemplary antigens that are over-expressed by tumor-associated neovasculature as compared to normal vasculature include, but are not limited to, VEGF/KDR, Tie2, vascular cell adhesion molecule (VCAM), endoglin and α₅β₃ integrin/vitronectin. Other antigens that are over-expressed by tumor-associated neovasculature as compared to normal vasculature are known to those of skill in the art and are suitable for targeting by the disclosed viruses.

(3) Chemokines/Chemokine Receptors

In another embodiment, the virus is engineered to express a domain that specifically binds to a chemokine or a chemokine receptor. Chemokines are soluble, small molecular weight (8-14 kDa) proteins that bind to their cognate G-protein coupled receptors (GPCRs) to elicit a cellular response, usually directional migration or chemotaxis. Tumor cells secrete and respond to chemokines, which facilitate growth that is achieved by increased endothelial cell recruitment and angiogenesis, subversion of immunological surveillance and maneuvering of the tumoral leukocyte profile to skew it such that the chemokine release enables the tumor growth and metastasis to distant sites. Thus, chemokines are vital for tumor progression.

Based on the positioning of the conserved two N-terminal cysteine residues of the chemokines, they are classified into four groups: CXC, CC, CX3C and C chemokines. The CXC chemokines can be further classified into ELR+ and ELR− chemokines based on the presence or absence of the motif ‘glu-leu-arg (ELR motif)’ preceding the CXC sequence. The CXC chemokines bind to and activate their cognate chemokine receptors on neutrophils, lymphocytes, endothelial and epithelial cells. The CC chemokines act on several subsets of dendritic cells, lymphocytes, macrophages, eosinophils, natural killer cells but do not stimulate neutrophils as they lack CC chemokine receptors except murine neutrophils. There are approximately 50 chemokines and only 20 chemokine receptors, thus there is considerable redundancy in this system of ligand/receptor interaction.

Chemokines elaborated from the tumor and the stromal cells bind to the chemokine receptors present on the tumor and the stromal cells. The autocrine loop of the tumor cells and the paracrine stimulatory loop between the tumor and the stromal cells facilitate the progression of the tumor. Notably, CXCR2, CXCR4, CCR2 and CCR7 play major roles in tumorigenesis and metastasis. CXCR2 plays a vital role in angiogenesis and CCR2 plays a role in the recruitment of macrophages into the tumor microenvironment. CCR7 is involved in metastasis of the tumor cells into the sentinel lymph nodes as the lymph nodes have the ligand for CCR7, CCL21. CXCR4 is mainly involved in the metastatic spread of a wide variety of tumors.

ii. Molecular Classes of Targeting Domains

(1) Ligands and Receptors

In one embodiment, tumor or tumor-associated neovasculature targeting domains are ligands that bind to cell surface antigens or receptors that are specifically expressed on tumor cells or tumor-associated neovasculature or are overexpressed on tumor cells or tumor-associated neovasculature as compared to normal tissue. Tumors also secrete a large number of ligands into the tumor microenvironment that affect tumor growth and development. Receptors that bind to ligands secreted by tumors, including, but not limited to, growth factors, cytokines and chemokines, including the chemokines discussed above, are suitable as targeting domains for the viruses disclosed herein. Ligands secreted by tumors can be targeted using soluble fragments of receptors that bind to the secreted ligands. Soluble receptor fragments are fragments of polypeptides that may be shed, secreted or otherwise extracted from the producing cells and include the entire extracellular domain, or fragments thereof.

(2) Single polypeptide antibodies

In another embodiment, tumor or tumor-associated neovasculature targeting domains are single polypeptide antibodies that bind to cell surface antigens or receptors that are specifically expressed on tumor cells or tumor-associated neovasculature or are overexpressed on tumor cells or tumor-associated neovasculature as compared to normal tissue.

(3) Fc domains

In another embodiment, tumor or tumor-associated neovasculature targeting domains are Fe domains of immunoglobulin heavy chains that bind to Fe receptors expressed on tumor cells or on tumor-associated neovasculature. As defined herein, the Fc region includes polypeptides containing the constant region of an antibody excluding the first constant region immunoglobulin domain. Thus Fc refers to the last two constant region immunoglobulin domains of IgA, IgD, and IgG, and the last three constant region immunoglobulin domains of IgE and IgM. In a preferred embodiment, the Fe domain is derived from a human or murine immunoglobulin. In a more preferred embodiment, the Fc domain is derived from human IgG1 or murine IgG2a including the C_{H}2 and C_{H}3 regions.

c. Therapeutic Proteins

Viruses can also be engineered to include one or more additional genes that encode a therapeutic protein. Suitable therapeutic proteins, such as cytokines or chemokines, are known in the art. Preferred cytokines include, but are not limited to, granulocyte macrophage colony stimulating factor (GM-CSF), tumor necrosis factor alpha (TNFα), tumor necrosis factor beta (TNFβ), macrophage colony stimulating factor (M-CSF), interleukin-1 (IL-1), interleukin-2 (IL-2), interleukin-4 (IL-4), interleukin-5 (IL-5), interleukin-6 (IL-6), interleukin-10 (IL-10), interleukin-12 (IL-12), interleukin-15 (IL-15), interleukin-21 (IL-21), interferon alpha (IFNα), interferon beta (IFNβ), interferon gamma (IFNγ), and IGIF, and variants and fragments thereof.

Suitable chemokines include, but are not limited to, an alpha-chemokine or a beta-chemokine, including, but not limited to, a C5a, interleukin-8 (IL-8), monocyte chemotactic protein 1 alpha (MIP1α), monocyte chemotactic protein 1 beta (MIP1β), monocyte chemoattractant protein 1 (MCP-1), monocyte chemoattractant protein 3 (MCP-3), platelet activating factor (PAFR), N-formyl-methionyl-leucyl-[³H]phenylalanine (FMLPR), leukotriene B₄, gastrin releasing peptide (GRP), RANTES, eotaxin, lymphotactin, IP10, I-309, ENA78, GCP-2, NAP-2 and MGSA/gro, and variants and fragments thereof.

d. Viruses Engineered to Deliver Vaccine Antigens

In some embodiments, the therapeutic virus is a vaccine vector. VSV viruses expressing foreign viral glycoproteins have shown promise as a vaccine vectors (Roberts, et al., J. Virol. 73:3723-3732 (1999), Rose, et al., Cell, 106:539-549 (2001), Jones, et al., Nat. Med. 11:786-790 (2005)). Additionally, recombinant VSVs are able to accommodate large inserts and multiple genes in their genomes. This ability to incorporate large gene inserts in replication-competent viruses offers advantages over other RNA virus vectors, such as those based on alphaviruses and poliovirus.

VSV viruses can be engineered to incorporated one or more nucleic acid sequences encoding one or more non-native immunogenic antigens. One or more native VSV genes may be truncated or deleted to create additional space for the sequence encoding the immunogenic antigen. When expressed by the VSV virus administered to a patient in need thereof, the immunogenic antigen produces prophylactic or therapeutic immunity against a diseases or disorder. Immunogenic antigens can be expressed as a fusion protein with other polypeptides including, but limited to, native VSV polypeptides, or as a non-fusion protein.

By way of non-limiting examples, the antigen can be protein or polypeptide derived from a virus, bacterium, parasite, plant, protozoan, fungus, tissue or transformed cell such as a cancer or leukemic cell. Antigens may be expressed as single antigens or may be provided in combination.

i. Viral Antigens

A viral antigen can be derived from any virus including, but not limited to, a virus from any of the following viral families: Arenaviridae, Arterivirus, Astroviridae, Baculoviridae, Badnavirus, Barnaviridae, Birnaviridae, Brornoviridae, Bunyaviridae, Caliciviridae, Capillovirus, Carlavirus, Caulimovirus, Circoviridae, Closterovirus, Comoviridae, Coronaviridae (e.g., Coronavirus, such as severe acute respiratory syndrome (SARS) virus), Corticoviridae, Cystoviridae, Deltavirus, Dianthovirus, Enamovirus, Filoviridae (e.g., Marburg virus and Ebola virus (e.g., Zaire, Reston, Ivory Coast, or Sudan strain)), Flaviviridae, (e.g., Hepatitis C virus, Dengue virus 1, Dengue virus 2, Dengue virus 3, and Dengue virus 4), Hepadnaviridae, Herpesviridae (e.g., Human herpesvirus 1, 3, 4, 5, and 6, and Cytomegalovirus), Hypoviridae, Iridoviridae, Leviviridae, Lipothrixviridae, Microviridae, Orthomyxoviridae (e.g., Influenzavirus A and B and C), Papovaviridae, Paramyxoviridae (e.g., measles, mumps, and human respiratory syncytial virus), Parvoviridae, Picornaviridae (e.g., poliovirus, rhinovirus, hepatovirus, and aphthovirus), Poxyiridae (e.g., vaccinia and smallpox virus), Reoviridae (e.g., rotavirus), Retroviridae (e.g., lentivirus, such as human immunodeficiency virus (HIV) 1 and HIV 2), Rhabdoviridae (for example, rabies virus, measles virus, respiratory syncytial virus, etc.), Togaviridae (for example, rubella virus, dengue virus, etc.), and Totiviridae. Suitable viral antigens also include all or part of Dengue protein M, Dengue protein E, Dengue DiNS1, Dengue D1NS2, and Dengue D1NS3.

Viral antigens may be derived from a particular strain such as a papilloma virus, a herpes virus, i.e. herpes simplex 1 and 2; a hepatitis virus, for example, hepatitis A virus (HAV), hepatitis B virus (HBV), hepatitis C virus (HCV), the delta hepatitis D virus (HDV), hepatitis E virus (HEV) and hepatitis G virus (HGV), the tick-borne encephalitis viruses; parainfluenza, varicella-zoster, cytomeglavirus, Epstein-Barr, rotavirus, rhinovirus, adenovirus, coxsackieviruses, equine encephalitis, Japanese encephalitis, yellow fever, Rift Valley fever, and lymphocytic choriomeningitis.

ii. Bacterial Antigens

Bacterial antigens can originate from any bacteria including, but not limited to, Actinomyces, Anabaena, Bacillus, Bacteroides, Bdellovibrio, Bordetella, Borrelia, Campylobacter, Caulobacter, Chlamydia, Chlorobium, Chromatium, Clostridium, Corynebacterium, Cytophaga, Deinococcus, Escherichia, Francisella, Halobacterium, Heliobacter, Haemophilus, Hemophilus influenza type B (HIB), Hyphomicrobium, Legionella, Leptspirosis, Listeria, Meningococcus A, B and C, Methanobacterium, Micrococcus, Myobacterium, Mycoplasma, Myxococcus, Neisseria, Nitrobacter, Oscillatoria, Prochloron, Proteus, Pseudomonas, Phodospirillum, Rickettsia, Salmonella, Shigella, Spirillum, Spirochaeta, Staphylococcus, Streptococcus, Streptomyces, Sulfolobus, Thermoplasma, Thiobacillus, and Treponema, Vibrio, and Yersinia.

iii. Parasite Antigens

Parasite antigens can be obtained from parasites such as, but not limited to, an antigen derived from Cryptococcus neoformans, Histoplasma capsulatum, Candida albicans, Candida tropicalis, Nocardia asteroides, Rickettsia ricketsii, Rickettsia typhi, Mycoplasma pneumoniae, Chlamydial psittaci, Chlamydial trachomatis, Plasmodium falciparum, Trypanosoma brucei, Entamoeba histolytica, Toxoplasma gondii, Trichomonas vaginalis and Schistosoma mansoni. These include Sporozoan antigens, Plasmodian antigens, such as all or part of a Circumsporozoite protein, a Sporozoite surface protein, a liver stage antigen, an apical membrane associated protein, or a Merozoite surface protein.

iv. Tumor Antigens

The antigen can be a tumor antigen, including a tumor-associated or tumor-specific antigen, such as, but not limited to, alpha-actinin-4, Bcr-Abl fusion protein, Casp-8, beta-catenin, cdc27, cdk4, cdkn2a, coa-1, dek-can fusion protein, EF2, ETV6-AML1 fusion protein, LDLR-fucosyltransferaseAS fusion protein, HLA-A2, HLA-A11, hsp70-2, KIAAO205, Mart2, Mum-1, 2, and 3, neo-PAP, myosin class I, OS-9, pml-RARα fusion protein, PTPRK, K-ras, N-ras, Triosephosphate isomeras, Bage-1, Gage 3,4,5,6,7, GnTV, Herv-K-mel, Lage-1, Mage-A1,2,3,4,6,10,12, Mage-C2, NA-88, NY-Eso-1/Lage-2, SP17, SSX-2, and TRP2-Int2, MelanA (MART-I), gp100 (Pmel 17), tyrosinase, TRP-1, TRP-2, MAGE-1, MAGE-3, BAGE, GAGE-1, GAGE-2, p15(58), CEA, RAGE, NY-ESO (LAGE), SCP-1, Hom/MeI-40, PRAME, p53, H-Ras, HER-2/neu, BCR-ABL, E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, p180erbB-3, c-met, nm-23H1, PSA, TAG-72-4, CA 19-9, CA 72-4, CAM 17.1, NuMa, K-ras, β-Catenin, CDK4, Mum-1, p16, TAGE, PSMA, PSCA, CT7, telomerase, 43-9F, 5T4, 791Tgp72, α-fetoprotein, 13HCG, BCA225, BTAA, CA 125, CA 15-3 (CA 27.29\BCAA), CA 195, CA 242, CA-50, CAM43, CD681\KP1, CO-029, FGF-5, G250, Ga733 (EpCAM), HTgp-175, M344, MA-50, MG7-Ag, MOV18, NB\70K, NY-CO-1, RCAS1, SDCCAG16, TA-90 (Mac-2 binding protein\cyclophilin C-associated protein), TAAL6, TAG72, TLP, and TPS.

2. Pharmaceutical Compositions

Immunizing and therapeutic viruses are typically administered to a patient in need thereof in a pharmaceutical composition. Pharmaceutical compositions containing virus may be for systemic or local administration, such as intratumoral. Dosage forms for administration by parenteral (intramuscular (IM), intraperitoneal (IP), intravenous (IV) or subcutaneous injection (SC)), or transmucosal (nasal, vaginal, pulmonary, or rectal) routes of administration can be formulated. In the most preferred embodiments, the immunizing virus is delivered peripherally by intranasally or by intramuscular injection, and the therapeutic virus is delivered by local injection, for example intracranial injection, preferably at the tumor site.

a. Effective Amounts

As generally used herein, an “effective amount” is that amount which is able to induce a desired result in a treated subject. The desired results will depend on the disease or condition to be treated. The precise dosage will vary according to a variety of factors such as subject-dependent variables (e.g., age, immune system health, etc.), the disease, and the treatment being effected. For example, an effective amount of immunizing virus generally results in production of antibody and/or activated T cells that kill or limit proliferation of or infection by a virus. An effective amount of the immunizing virus preferably is sufficient to reduce neurovirulence of the therapeutic virus compared to administration of the therapeutic virus without first administering the immunizing virus. Therapeutically effective amounts of the therapeutic viruses disclosed herein used in the treatment of cancer will generally kill tumor cells or inhibit proliferation or metastasis of the tumor cells. Symptoms of cancer may be physical, such as tumor burden, or biological such as proliferation of cancer cells. The actual effective amounts of virus can vary according to factors including the specific virus administered, the particular composition formulated, the mode of administration, and the age, weight, condition of the subject being treated, as well as the route of administration and the disease or disorder.

b. Dosages

Appropriate dosages can be determined by a person skilled in the art, considering the therapeutic context, age, and general health of the recipient. The selected dosage depends upon the desired therapeutic effect, on the route of administration, and on the duration of the treatment desired. Active virus can also be measured in terms of plaque-forming units (PFU). A plaque-forming unit can be defined as areas of cell lysis (CPE) in monolayer cell culture, under overlay conditions, initiated by infection with a single virus particle. Generally dosage levels of virus between 10² and 10¹² PFU are administered to humans. Virus is typically administered in a liquid suspension, in a volume ranging between 10 μl and 100 ml depending on the route of administration. Generally, dosage and volume will be lower for intratumoral injection as compared to systemic administration or infusion. The dose may be administered once or multiple times. Typically the dose will be 100 μl administered intratumorly in multiple doses, while systemic or regional administration via subcutaneous, intramuscular, intra-organ, or intravenous administration will be from 10 to 100 ml.

The term “pharmaceutically acceptable” means a non-toxic material that does not interfere with the effectiveness of the biological activity of the active ingredients. The term “pharmaceutically-acceptable carrier” means one or more compatible solid or liquid fillers, diluents or encapsulating substances which are suitable for administration to a human or other vertebrate animal. The term “carrier” refers to an organic or inorganic ingredient, natural or synthetic, with which the active ingredient is combined to facilitate the application.

Pharmaceutical compositions may be formulated in a conventional manner using one or more physiologically acceptable carriers including excipients and auxiliaries which facilitate processing of the active compounds into preparations which can be used pharmaceutically. The compositions may be administered in combination with one or more physiologically or pharmaceutically acceptable carriers, thickening agents, co-solvents, adhesives, antioxidants, buffers, viscosity and absorption enhancing agents and agents capable of adjusting osmolarity of the formulation. Proper formulation is dependent upon the route of administration chosen. If desired, the compositions may also contain minor amount of nontoxic auxiliary substances such as wetting or emulsifying agents, dyes, pH buffering agents, or preservatives. The formulations should not include membrane disrupting agents which could kill or inactivate the virus.

i. Formulations for Local or Parenteral Administration

In a preferred embodiment, compositions including oncolytic virus disclosed herein, are administered in an aqueous solution, by parenteral injection. Injection includes, but it not limited to, local, intratumoral, intravenous, intraperitoneal, intramuscular, or subcutaneous. The formulation may also be in the form of a suspension or emulsion. In general, pharmaceutical compositions are provided including effective amounts of virus, and optionally include pharmaceutically acceptable diluents, preservatives, solubilizers, emulsifiers, adjuvants and/or carriers. Such compositions include diluents such as sterile water, buffered saline of various buffer content (e.g., Tris-HCl, acetate, phosphate), pH and ionic strength; and optionally, additives such as anti-oxidants (e.g., ascorbic acid, sodium metabisulfite), and preservatives and bulking substances (e.g., lactose, mannitol). Examples of non-aqueous solvents or vehicles are propylene glycol, polyethylene glycol, vegetable oils, such as olive oil and corn oil, gelatin, and injectable organic esters such as ethyl oleate. A preferred solution is phosphate buffered saline or sterile saline.

ii. Formulations for Mucosal Administration

In some embodiments, the compositions are formulated for mucosal administration, such as through nasal, pulmonary, or buccal delivery.

Mucosal formulations may include one or more agents for enhancing delivery through the nasal mucosa. Agents for enhancing mucosal delivery are known in the art, see, for example, U.S. Patent Application No. 20090252672 to Eddington, and U.S. Patent Application No. 20090047234 to Touitou. Acceptable agents include, but are not limited to, chelators of calcium (EDTA), inhibitors of nasal enzymes (boro-leucin, aprotinin), inhibitors of muco-ciliar clearance (preservatives), solubilizers of nasal membrane (cyclodextrin, fatty acids, surfactants) and formation of micelles (surfactants such as bile acids, Laureth 9 and taurodehydrofusidate (STDHF)). Compositions may include one or more absorption enhancers, including surfactants, fatty acids, and chitosan derivatives, which can enhance delivery by modulation of the tight junctions (TJ) (B. J. Aungst, et al., J. Pharm. Sci. 89(4):429-442 (2000)). In general, the optimal absorption enhancer should possess the following qualities: its effect should be reversible, it should provide a rapid permeation enhancing effect on the cellular membrane of the mucosa, and it should be non-cytotoxic at the effective concentration level and without deleterious and/or irreversible effects on the cellular or virus membrane. Intranasal compositions maybe administered using devices known in the art, for example a nebulizer.

B. Peripheral Administration of Immunizing Virus

It has been discovered that one or more peripheral administrations with an immunizing virus elicits an adaptive immune response that protects the brain from potential side-effects of oncolytic virus therapy. As described above, the term “immunizing virus” includes live virus as well as viral subunits, proteins and fragments thereof, antigenic polypeptides, nucleic acids, and expression vectors containing nucleic acids encoding viral subunits, proteins, or fragments thereof, or antigenic polypeptides which can be useful in eliciting an immune response. For example, if the immunizing virus is a VSV virus, the immunizing virus includes, but is not limited to, live VSV virus, or the N, P, M, G, or L proteins, or combinations thereof.

The immunizing virus may be the same virus, or a different virus than the therapeutic virus. The immunizing virus should initiate an adaptive immune response that is sufficient to attenuate, reduce, or prevent the neurovirulence of the therapeutic virus. The therapeutic virus administered after a first administration of immunizing virus should have reduced neurovirulence compared to therapeutic virus administered without a first administration of immunizing virus. In preferred embodiments, the immunizing virus is similar to the therapeutic virus. For example if the therapeutic virus is a VSV, the immunizing virus is preferably a VSV, or an antigenic protein or nucleic acid component thereof. In some embodiments the immunizing virus has an attenuated phenotype compared to the therapeutic virus. As described above, suitable immunizing viruses include wildtype viruses, as well as mutant and variants thereof. In one preferred embodiment, the immunizing virus is a wildtype virus, while the therapeutic virus is a mutant or variant of the same virus that has attenuated neurovirulence. In some embodiments, therapeutic viruses may be engineered to express therapeutic proteins or targeting molecules. Immunizing viruses may also be engineered to express additional proteins, but preferably are not. As described in the examples below, VSV-G/GFP is a suitable immunizing virus. The nucleotide sequence for VSV-G/GFP is GenBank Accession H478454. A link to the sequence is http://www.ncbi.nlm.nih.gov/nuccore/218457792.

Immunizing viruses are administered sufficiently prior to therapeutic viruses to elicit an adaptive immune response. Immunizing viruses are typically administered one or more times at least about 5 days, preferably 1 week, more preferably greater than one week before administration of the therapeutic virus. Immunizing viruses can be administered up to 1, 2, 3, 4, 5, or more weeks before the therapeutic virus. Immunizing viruses can be administered up to 1, 2, 3, 4, 5, or more months before the therapeutic virus. Most preferably the immunizing virus is administered between about ten days and 12 weeks before the therapeutic virus.

After an initial administration of the immunizing virus, subsequent booster immunizations can be administered. For example, it may be desirable to administer the immunizing virus two or more times. A first administration of the immunizing virus is typically provided to a patient in need therefore prior to a first administration of the therapeutic virus. Subsequent administrations of the immunizing virus may occur before and/or after a first administration of the therapeutic virus. In preferred embodiments the immunizing virus is administered two or more times before the first administration of the therapeutic virus. In a non-limiting example, the immunizing virus is first administered on day 1, a booster of immunizing virus is administered six weeks later on day 43, and the therapeutic virus is first administered two weeks later on day 57.

Various factors may be considered when determining the frequency, dosage, duration, and number of administrations of immunizing virus, as well as the duration between administration of the immunizing virus and first administration of therapeutic virus. For example, the subject's adaptive immune response can be monitored to assess the effectiveness of the immunization. Methods of measuring adaptive immune activation are known in the art and include antibody profiling, serum analysis for changes in levels of antibodies, cytokines, chemokines, or other inflammatory molecules, and cell counts and/or cell profiling using extracellular markers to assess the numbers and types of immune cells such as B cells and T cells.

Immunizing virus is always delivered to a subject in need thereof by peripheral administration, and not directly or locally to the site in need of treatment by therapeutic virus. Peripheral administration includes intravenous, by injection or infusion, intraperitoneal, intramuscular, subcutaneous, and mucosal such as intranasal delivery. In some embodiments, the composition is delivered systemically, by injection into the circulatory system (i.e. intravenous) or an appropriate lymphoid tissue, such as the spleen, lymph nodes or mucosal-associated lymphoid tissue. The injections can be given at one, or multiple locations. Preferably the immunizing virus is administered intranasally or by intramuscular injection, most preferably by intranasal delivery.

Generally immunizing virus is administered to humans at dosage levels between 10² and 10¹² PFU. Virus is typically administered in a liquid suspension, in a volume ranging between 10 μl and 100 ml depending on the route of administration.

It may also be desirable to administer the immunizing virus in combination with one or more adjuvants. These can be incorporated into, administered with, or administered separately from, the immunogenizing virus. Depending on whether or not the individual is a human or an animal, the adjuvant can be, but is not limited to, one or more of the following: oil emulsions (e.g., Freund's adjuvant); saponin formulations; virosomes and viral-like particles; bacterial and microbial derivatives; immunostimulatory oligonucleotides; ADP-ribosylating toxins and detoxified derivatives; alum; BCG; mineral-containing compositions (e.g., mineral salts, such as aluminium salts and calcium salts, hydroxides, phosphates, sulfates, etc.); bioadhesives and/or mucoadhesives; microparticles; liposomes; polyoxyethylene ether and polyoxyethylene ester formulations; polyphosphazene; muramyl peptides; imidazoquinolone compounds; and surface active substances (e.g. lysolecithin, pluronic polyols, polyanions, peptides, oil emulsions, keyhole limpet hemocyanin, and dinitrophenol).

C. Administration of Therapeutic Virus

As described in the examples below, suitable therapeutic viruses may include VSV-CT9-M51, VSV-CT1, VSV-CT9, and VSV-G/GFP. In a preferred embodiment the therapeutic virus is VSV-CT9-M51, or variant thereof. The number, frequency, and duration with which therapeutic viruses are administered will depend on the condition to be treated. Pharmaceutical compositions including therapeutic viruses may be administered once or more than once, for example 2, 3, 4, 5, or more times. Serial administration of therapeutic virus may occur days, weeks, or months apart. Boosters of immunizing virus may be administered between therapeutic treatments. It may be particularly preferable to administer a booster of immunizing virus if there are lengthy delays between treatments with therapeutic virus, for example, one or more years.

Virus can be administered peripherally, or could be injected directly into the tumor within the brain. In addition, virus could be used after resection of the main body of the tumor, by adding directly after surgery, or after a period of one to two weeks to allow recovery of local damage. Adding virus after surgical resection would eliminate any remaining tumor cells that the neurosurgeon did not remove. The injections can be given at one, or multiple locations. As described in the Examples below, attenuated therapeutic virus administered systemically to mice was able to target and kill human glioblastoma transplanted into mouse brains.

Generally virus is administered to humans at dosage levels between 10² and 10¹² PFU. Virus delivered locally, is typically administered in lower doses than virus administered systemically. When administered locally, therapeutic virus is administered to humans at dosage levels between 10² and 10⁶ PFU. Therapeutic virus is typically administered in a liquid suspension, in a low volume. The volume for local administration can range from about 20 nl to about 200 μl.

In some embodiments, it may be desirable to administer the therapeutic virus after or in combination with an immunosuppressant. Treatment with an immunosuppressant during administration with a therapeutic virus allows controlled suppression of the subject's immune system during administration of the therapeutic virus. This may be desirable, for example, if the capacity of the oncolytic virus to kill cancer is reduced due to an earlier administration of the immunizing virus. Treatment with the immunosuppressant is typically transient, and occurs during administration of the virus, particularly when the virus is being used to treat tumors and/or cancer. Following treatment with the therapeutic virus, treatment with the immunosuppressant is discontinued and the patient's immunity returns. The duration of immunosuppressive treatment will depend on the condition to be treated. Typically the immunosuppressive treatment will be long enough for the oncolytic virus to kill cancer cells, reduce tumor size, or inhibit tumor progression.

Suitable immunosuppressants are known in the art and include glucocorticoids, cytostatics (such as alkylating agents, antimetabolites, and cytotoxic antibodies), antibodies (such as those directed against T-cell recepotors or I1-2 receptors), drugs acting on immunophilins (such as cyclosporine, tacrolimus, and sirolimus) and other drugs (such as interferons, opioids, TNF binding proteins, mycophenolate, and other small molecules such as fingolimod). The dosage ranges for immunosuppressant agents are known in the art. The specific dosage will depend upon the desired therapeutic effect, the route of administration, and on the duration of the treatment desired. For example, when used as an immunosuppressant, a cytostatic maybe administered at a lower dosage than when used in chemotherapy. Immunosuppressants include, but are not limited to, FK506, prednisone, methylprednisolone, cyclophosphamide, thalidomide, azathioprine, and daclizumab, physalin B, physalin F, physalin G, seco-steroids purified from Physalis angulata L., 15-deoxyspergualin, MMF, rapamycin and its derivatives, CCl-779, FR 900520, FR 900523, NK86-1086, depsidomycin, kanglemycin-C, spergualin, prodigiosin25-c, cammunomicin, demethomycin, tetranactin, tranilast, stevastelins, myriocin, gliotoxin, FR 651814, SDZ214-104, bredinin, WS9482, mycophenolic acid, mimoribine, misoprostol, OKT3, anti-IL-2 receptor antibodies, azasporine, leflunomide, mizoribine, azaspirane, paclitaxel, altretamine, busulfan, chlorambucil, ifosfamide, mechlorethamine, melphalan, thiotepa, cladribine, fluorouracil, floxuridine, gemcitabine, thioguanine, pentostatin, methotrexate, 6-mercaptopurine, cytarabine, carmustine, lomustine, streptozotocin, carboplatin, cisplatin, oxaliplatin, iproplatin, tetraplatin, lobaplatin, JM216, JM335, fludarabine, aminoglutethimide, flutamide, goserelin, leuprolide, megestrol acetate, cyproterone acetate, tamoxifen, anastrozole, bicalutamide, dexamethasone, diethylstilbestrol, bleomycin, dactinomycin, daunorubicin, doxirubicin, idarubicin, mitoxantrone, losoxantrone, mitomycin-c, plicamycin, paclitaxel, docetaxel, topotecan, irinotecan, 9-amino camptothecan, 9-nitro camptothecan, GS-211, etoposide, teniposide, vinblastine, vincristine, vinorelbine, procarbazine, asparaginase, pegaspargase, octreotide, estramustine, and hydroxyurea, and combinations thereof. Preferred immunosuppressants will preferentially reduce or inhibit the subject's immune response, without reducing or inhibiting the activity of the virus.

The dosage ranges for immunosuppressant agents are known in the art. The specific dosage will depend upon the desired therapeutic effect, the route of administration, and on the duration of the treatment desired. For example, when used as an immunosuppressant, a cytostatic maybe administered at a lower dosage than when used in chemotherapy.

D. Subjects to be Treated

1. Cancer and Tumor Therapy

In general, the disclosed methods for attenuating the neurovirulence of a therapeutic virus are useful in the context of cancer, including tumor therapy, particular brain tumor therapy. Therefore, in some embodiments, the therapeutic virus is an oncolytic virus used to treat a tumor.

In a mature animal, a balance usually is maintained between cell renewal and cell death in most organs and tissues. The various types of mature cells in the body have a given life span; as these cells die, new cells are generated by the proliferation and differentiation of various types of stem cells. Under normal circumstances, the production of new cells is so regulated that the numbers of any particular type of cell remain constant. Occasionally, though, cells arise that are no longer responsive to normal growth-control mechanisms. These cells give rise to clones of cells that can expand to a considerable size, producing a tumor or neoplasm. A tumor that is not capable of indefinite growth and does not invade the healthy surrounding tissue extensively is benign. A tumor that continues to grow and becomes progressively invasive is malignant. The term cancer refers specifically to a malignant tumor. In addition to uncontrolled growth, malignant tumors exhibit metastasis. In this process, small clusters of cancerous cells dislodge from a tumor, invade the blood or lymphatic vessels, and are carried to other tissues, where they continue to proliferate. In this way a primary tumor at one site can give rise to a secondary tumor at another site.

The compositions and methods described herein are useful for treating subjects having benign or malignant tumors by delaying or inhibiting the growth of a tumor in a subject, reducing the growth or size of the tumor, inhibiting or reducing metastasis of the tumor, and/or inhibiting or reducing symptoms associated with tumor development or growth. The examples below demonstrate that the methods disclosed herein are useful in attenuating the neurovirulence VSV viruses while maintaining their oncolytic potential for treating brain tumors in vivo.

Malignant tumors which may be treated are classified herein according to the embryonic origin of the tissue from which the tumor is derived. Carcinomas are tumors arising from endodermal or ectodermal tissues such as skin or the epithelial lining of internal organs and glands. The disclosed compositions are particularly effective in treating carcinomas. Sarcomas, which arise less frequently, are derived from mesodermal connective tissues such as bone, fat, and cartilage. The leukemias and lymphomas are malignant tumors of hematopoietic cells of the bone marrow. Leukemias proliferate as single cells, whereas lymphomas tend to grow as tumor masses. Malignant tumors may show up at numerous organs or tissues of the body to establish a cancer.

The types of cancer that can be treated with the provided compositions and methods include, but are not limited to, cancers such as vascular cancer such as multiple myeloma, adenocarcinomas and sarcomas, of bone, bladder, brain, breast, cervical, colo-rectal, esophageal, kidney, liver, lung, nasopharangeal, pancreatic, prostate, skin, stomach, and uterine. In some embodiments, the disclosed compositions are used to treat multiple cancer types concurrently. The compositions can also be used to treat metastases or tumors at multiple locations.

The disclosed methods are particularly useful in treating brain tumors. Brain tumors include all tumors inside the cranium or in the central spinal canal. They are created by an abnormal and uncontrolled cell division, normally either in the brain itself (neurons, glial cells (astrocytes, oligodendrocytes, ependymal cells, myelin-producing Schwann cells), lymphatic tissue, blood vessels), in the cranial nerves, in the brain envelopes (meninges), skull, pituitary and pineal gland, or spread from cancers primarily located in other organs (metastatic tumors). “Primary” brain tumors originate in the brain and “secondary” (metastatic) brain tumors originate from cancer cells that have migrated from other parts of the body. Primary brain cancer rarely spreads beyond the central nervous system, and death results from uncontrolled tumor growth within the limited space of the skull. Metastatic brain cancer indicates advanced disease and has a poor prognosis. Primary brain tumors can be cancerous or noncancerous. Both types take up space in the brain and may cause serious symptoms (e.g., vision or hearing loss) and complications (e.g., stroke). All cancerous brain tumors are life threatening (malignant) because they have an aggressive and invasive nature. A noncancerous primary brain tumor is life threatening when it compromises vital structures (e.g., an artery).

Brain tumors include all tumors inside the cranium or in the central spinal canal. They are created by an abnormal and uncontrolled cell division, normally either in the brain itself (neurons, glial cells (astrocytes, oligodendrocytes, ependymal cells, myelin-producing Schwann cells), lymphatic tissue, blood vessels), in the cranial nerves, in the brain envelopes (meninges), skull, pituitary and pineal gland, or spread from cancers primarily located in other organs (metastatic tumors). Examples of brain tumors include, but are not limited to oligodendroglioma, meningioma, supratentorial ependymona, pineal region tumors, medulloblastoma, cerebellar astrocytoma, infratentorial ependymona, brainstem glioma, schwannomas, pituitary tumors, craniopharyngioma, optic glioma, and astrocytoma.

2. Vaccination

In general, the disclosed methods for attenuating the neurovirulence of a therapeutic virus are also useful in the context of VSV vaccine delivery. In some embodiments, the therapeutic virus is a vaccine vector. As described above, VSV can be engineered to express one or more immunogenic antigens. Expression of these antigens in a patient in need thereof presents the antigen to the immune system and provokes an immune response. Vaccines can be administered prophylactically or therapeutically. Vaccines can also be administered according to a vaccine schedule. A vaccine schedule is a series of vaccinations, including the timing of all doses. Many vaccines require multiple doses for maximum effectiveness, either to produce sufficient initial immune response or to boost response that fades over time. Vaccine schedules are known in the art, and are designed to achieve maximum effectiveness. The adaptive immune response can be monitored using methods known in the art to measure the effectiveness of the vaccination protocol.

E. Combination Therapies

Administration of the disclosed compositions containing oncolytic viruses may be coupled with surgical, radiologic, other therapeutic approaches to treatment of tumors.

1. Surgery

The disclosed compositions and methods can be used as an adjunct to surgery. Surgery is a common treatment for many types of benign and malignant tumors. As it is often not possible to remove all the tumor cells from during surgery, the disclosed compositions containing oncolytic virus are particularly useful subsequent to resection of the primary tumor mass, and would be able to infect and destroy even dispersed tumor cells.

In a preferred embodiment, the disclosed compositions and methods are used as an adjunct or alternative to neurosurgery. The compositions are particularly well suited to treat areas of the brain that is difficult to treat surgically, for instance high grade tumors of the brain stem, motor cortex, basal ganglia, or internal capsule. High grade gliomas in these locations are generally considered inoperable. An additional situation where an oncolytic virus may be helpful is in regions where the tumor is either wrapped around critical vasculature, or in an area that is difficult to treat surgically.

2. Therapeutic Agents

The viral compositions can be administered to a subject in need thereof alone or in combination with one or more additional therapeutic agents selected based on the condition, disorder or disease to be treated. A description of the various classes of suitable pharmacological agents and drugs may be found in Goodman and Gilman, The Pharmacological Basis of Therapeutics, (11th Ed., McGraw-Hill Publishing Co.) (2005).

Additional therapeutic agents include conventional cancer therapeutics such as chemotherapeutic agents, cytokines, chemokines, and radiation therapy. The majority of chemotherapeutic drugs can be divided into: alkylating agents, antimetabolites, anthracyclines, plant alkaloids, topoisomerase inhibitors, and other antitumour agents. All of these drugs affect cell division or DNA synthesis and function in some way. Additional therapeutics include monoclonal antibodies and the tyrosine kinase inhibitors e.g. imatinib mesylate (GLEEVEC® or GLIVEC®), which directly targets a molecular abnormality in certain types of cancer (chronic myelogenous leukemia, gastrointestinal stromal tumors).

Representative chemotherapeutic agents include, but are not limited to, cisplatin, carboplatin, oxaliplatin, mechlorethamine, cyclophosphamide, chlorambucil, vincristine, vinblastine, vinorelbine, vindesine, taxol and derivatives thereof, irinotecan, topotecan, amsacrine, etoposide, etoposide phosphate, teniposide, epipodophyllotoxins, trastuzumab (HERCEPTIN®), cetuximab, and rituximab (RITUXAN® or MASTHERA®), bevacizumab (AVASTIN®), and combinations thereof.

Preferred chemotherapeutics will affect tumors or cancer cells, without diminishing the activity of the virus. For example, in a preferred embodiment, the additional therapeutic agent inhibits proliferation of cancer cells without affecting targeting, infectivity, or replication of the virus.

a. Anticancer Agents

The compositions can be administered with an antibody or antigen binding fragment thereof specific for growth factor receptors or tumor specific antigens. Representative growth factors receptors include, but are not limited to, epidermal growth factor receptor (EGFR; HER1); c-erbB2 (HER2); c-erbB3 (HER3); c-erbB4 (HER4); insulin receptor; insulin-like growth factor receptor 1 (IGF-1R); insulin-like growth factor receptor 2/Mannose-6-phosphate receptor (IGF-II RIM-6-P receptor); insulin receptor related kinase (IRRK); platelet-derived growth factor receptor (PDGFR); colony-stimulating factor-1receptor (CSF-1R) (c-Fms); steel receptor (c-Kit); Flk2/Flt3; fibroblast growth factor receptor 1 (Flg/Cek1); fibroblast growth factor receptor 2 (Bek/Cek3/K-Sam); Fibroblast growth factor receptor 3; Fibroblast growth factor eceptor 4; nerve growth factor receptor (NGFR) (TrkA); BDNF receptor (TrkB); NT-3-receptor (TrkC); vascular endothelial growth factor receptor 1 (FM); vascular endothelial growth factor receptor 2/Flk1/KDR; hepatocyte growth factor receptor (HGF-R/Met); Eph; Eck; Eek; Cek4/Mek4/HEK; Cek5; Elk/Cek6; Cek7; Sek/Cek8; Cek9; Cek10; HEK11; 9 Ror1; Ror2; Ret; Ax1; RYK; DDR; and Tie.

b. Therapeutic Proteins

It may be desirable to administer the disclosed compositions in combination with therapeutic proteins. VSV is an effective oncolytic virus, in-part, by taking advantage of defects in the interferon system. Administration of therapeutic proteins such as IFN-α, or IFN-0113 pathway inducer polyriboinosinic polyribocytidylic acid [poly(I:C)] are effective in protecting normal cells from the oncolytic activity, while leaving the tumor cells susceptible to infection and death (Wollmann, et al. J. Vivol., 81(3): 1479-1491 (2007). Therefore, in some embodiments, the disclosed compositions are administered in combination with a therapeutic protein to reduce infectivity and death of normal cells. Suitable therapeutic proteins are described above.

c. Immunosuppressants

Immunosuppressants such as those discussed above, preferably cyclosporin, prednisone, dexamethasone, or other steroidal anti-inflammatory, can be used to reduce the immune response immediately before, during, or shortly after administration of the therapeutic virus. This allows maximum infection and killing by the therapeutic virus. The immunosuppressant is then discontinued or decreased to allow the patient's immune system to prevent inflammation and/or killing of the virus after it has competed the desired killing of tumor or diseased tissue.

F. Kits

Dosage units include virus in a pharmaceutically acceptable carrier for shipping and storage and/or administration. Active virus should be shipped and stored using a method consistent with viability such as in cooler containing dry ice so that cells are maintained below 4° C., and preferably below −20° C. VSV virus should not be lyophilized. Components of the kit may be packaged individually and can be sterile. In one embodiment, a pharmaceutically acceptable carrier containing an effective amount of virus is shipped and stored in a sterile vial. The sterile vial may contain enough virus for one or more doses. Virus may be shipped and stored in a volume suitable for administration, or may be provided in a concentrated titer that is diluted prior to administration. In another embodiment, a pharmaceutically acceptable carrier containing an effective amount of virus can be shipped and stored in a syringe.

Typical concentrations of viral particles in the sterile saline, phosphate buffered saline or other suitable media for the virus is in the range of 10⁸ to 10⁹ with a maximum of 10¹². Dosage units should not contain membrane disruptive agents nor should the viral solution be frozen and dried (i.e., lyophilized), which could kill the virus.

Kits containing syringes of various capacities or vessels with deformable sides (e.g., plastic vessels or plastic-sided vessels) that can be squeezed to force a liquid composition out of an orifice are provided. The size and design of the syringe will depend on the route of administration. For example, in one embodiment, a syringe for administering virus intratumorally, is capable of accurately delivering a smaller volume (such as 1 to 100 μl). Typically, a larger syringe, pump or catheter will be used to administer virus systemically. Any of the kits can include instructions for use.

### III. Methods of Manufacture

A. Engineering Recombinant VSV Viruses

The VSV genome is a single negative-sense, non-segmented stand of RNA that contains five genes (N, L, P, M, and G) and has a total size of 11.161 kb. Methods of engineering recombinant viruses by reconstituting VSV from DNA encoding a positive-sense stand of RNA are known in the art (Lawson, et al., PNAS, 92:4477-4481 (1995), Dalton and Rose, Virology, 279:414-421 (2001)). For example, recombinant DNA can be transcribed by T7 RNA polymerase to generate a full-length positive-strand RNA complimentary to the viral genome. Expression of this RNA in cells also expressing the VSV nucleocapsid protein and the two VSV polymerase subunits results in production of VSV virus (Lawson, et al., PNAS, 92:4477-4481 (1995)). In this way, VSV viruses can be engineered to express variant proteins, additional proteins, foreign antigens, targeting proteins, or therapeutic proteins using known cloning methods.

B. Creating Mutant VSV Virus

RNA viruses are prone to spontaneous genetic variation. The mutation rate of VSV is about 10⁻⁴ per nucleotide replicated, which is approximately one nucleotide change per genome (Drake, et al, Proc. Natl. Acad. Sci. USA, 96:13910-13913). Therefore, mutant VSV viruses exhibiting desired properties can be developed by applying selective pressure. Methods for adaption of VSV viruses through repeated passaging is described in the art. See, for example, Wollmann, et al., J. Virol., 79(10): 6005-6022 (2005). Selective pressure can be applied by repeated passaging and enhanced selection to create mutant virus with desirable traits such as increased infectivity and oncolytic potential for a cell type of interest. The cell type of interest could be general, such as cancer cells, or specific such as glioblastoma cells. Mutant virus can also be selected based on reduced toxicity to normal cells. Methods of enhanced selection include, but are not limited to, short time for viral attachment to cells, collection of early viral progeny, and preabsorption of viral particles with high affinity of undesirable cells (such as normal cells). Mutations can be identified by sequencing the viral genome and comparing the sequence to the sequence of the parental strain.

DNA encoding the VSV genome can also be used as a substrate for random or site directed mutagenesis to develop VSV mutant viruses. Mutagenesis can be accomplished by a variety of standard, mutagenic procedures. Changes in single genes may be the consequence of point mutations that involve the removal, addition or substitution of a single nucleotide base within a DNA sequence, or they may be the consequence of changes involving the insertion or deletion of large numbers of nucleotides.

Mutations can arise spontaneously as a result of events such as errors in the fidelity of nucleic acid replication or the movement of transposable genetic elements (transposons) within the genome. They also are induced following exposure to chemical or physical mutagens. Such mutation-inducing agents include ionizing radiations, ultraviolet light and a diverse array of chemicals such as alkylating agents and polycyclic aromatic hydrocarbons all of which are capable of interacting either directly or indirectly (generally following some metabolic biotransformations) with nucleic acids. The nucleic acid lesions induced by such environmental agents may lead to modifications of base sequence when the affected DNA is replicated or repaired and thus to a mutation. Mutation also can be site-directed through the use of particular targeting methods. Various types of mutagenesis such as random mutagenesis, e.g., insertional mutagenesis, chemical mutagenesis, radiation mutagenesis, in vitro scanning mutagenesis, random mutagenesis by fragmentation and reassembly, and site specific mutagenesis, e.g., directed evolution, are described in U.S. Patent Application No. 2007/0026012.

Mutant viruses can be prepared by site specific mutagenesis of nucleotides in the DNA encoding the protein, thereby producing DNA encoding the mutant. Techniques for making substitution mutations at predetermined sites in DNA having a known sequence are well known, for example M13 primer mutagenesis and PCR mutagenesis. Amino acid substitutions are typically of single residues, but can occur at a number of different locations at once. Insertions usually will be on the order of about from 1 to 10 amino acid residues; and deletions will range about from 1 to 30 residues. Substitutions, deletions, insertions or any combination thereof can be combined to arrive at a final construct. The mutations must not place the sequence out of reading frame and preferably will not create complementary regions that could produce secondary mRNA structure. Substitutional variants are those in which at least one residue has been removed and a different residue inserted in its place.

### EXAMPLES

### Example VSV-CT1 and VSV-CT9-M51 Result in Longer Survival after Intracerebral Injections in Young Mice

Materials and Methods

Viruses

A number of VSV mutants have been described (Clarke, et al., J. Virol., 81:2056-64 (2007), Flanagan, et al., J. Virol., 77:5740-5748 (2003), Johnson, et al., Virology, 360:36-49 (2007), Simon, et al., J. Virol., 81:2078-82 (2007), Stojdl, et al., Cancer Cell, 4:263-275 (2003). Attenuation of VSV phenotype was accomplished by using either a virus with the cytoplasmic portion of the G protein truncated from 29 amino acids to a single amino acid (VSV-CT1), or by combining an M51 mutation with a partially truncated G protein reduced to 9 cytoplasmic amino acids. Both of these recombinant VSVs have previously been reported to show an attenuated phenotype after peripheral administration (Ahmed, et al., J. Virol. 82:9273-77 (2008), Publicover, J. Virol. 80:7028-36 (2006), Stojdl, et al., Cancer Cell, 4:263-275 (2003)).

Three VSVs were used in the experiments below. These three viruses have the following genotypes. 1. VSV-G/GFP—an expression cassette with a GFP sequence fused to the VSV-G was inserted between the wild-type G and L gene (Dalton, et al., Virology, 279:414-421 (2001), van den Poi, et al., J. Virol. 76:1309-1327 (2002)). 2. VSV-CT1—the cytoplasmic tail of the G-protein was truncated to one amino acid down from 29 amino acids. 3. VSV-CT9-M51—Position 51 of the M gene coding for methionine was deleted and the cytoplasmic tail of VSV-G was reduced from 29 to 9 amino acids. Generation of recombinant VSV strains are described in detail elsewhere (Dalton, et al., Virology, 279:414-421 (2001), Lawson, et al., Proc. Natl., Acad. Sci., 92:4477-4481 (1995), Publicover, et al., J. Virol. 80:7028-36 (2006), Schnell, et al., EMBO J. 17:1289-96 (1998), (van den Pol, et al., J. Comp., Neural. 516:456-481 (2009) and were kindly provided by Dr. J. Rose.

Animal Procedures

Animal experiments and postoperative care were performed in accordance with institutional guidelines of the Yale University Animal Care and Use Committee. Normal Swiss-Webster mice and immunodeficient homozygous CB17-SCID (CB17SC-M) were obtained from Taconic Inc. Mice were anesthetized by i.p. injection of a combination of ketamine and xylazine (100 and 10 mg/kg, respectively). Stereotactic intracerebral injections of viruses were performed in 4-5 week old animals into the left frontal lobe (1.5 mm lateral, 2 mm rostral to Bregma at 2 mm depth). For tumor implants 3×10⁵ tumor cells were injected bilaterally into the striatum (2 mm lateral, 0.4 mm rostral to Bregma at 3 mm depth). Intranasal virus inoculations were performed under light ketamine-xylazine anesthesia (50 mg/kg and 5 mg/kg respectively) with a 25 ul inoculum into each nostril.

Animals were immunized through either intranasal or intramuscular routes. Intranasal dose was 2.5×10⁷ PFU VSV-G/GFP given in 25 ul in each naris. The same dose was given 6 weeks later as a boost. Intracranial viral challenge was given 2 weeks after the boost dose.

Intracerebral injections in adult animals were given as a single stereotactic injection of VSV-CT9-M51 or VSV-CT1 in 200 nl into the left frontal lobe using a 1 ul Hamilton syringe. The injected dose equivalent was plaque assayed on the same day and contained either 15,000±2,000 PFU of VSV-CT9-M51 or VSV-CT1.

Sixteen-day-old animals were injected intracerebrally using a 1 ul Hamilton syringe into the forebrain at the left midpupillary line, 2 mm posterior to the eye at 2 mm depth from the skin perpendicular to the skull surface. Each virus injection contained either 8500 PFU of VSV-CT9-M51 or 8500 PFU of VSV-G/GFP in 200 nl (n=7 each). Any mouse showing serious neurological dysfunction was given an anesthetic overdose, and were considered to show a lethal effect of the virus.

Results

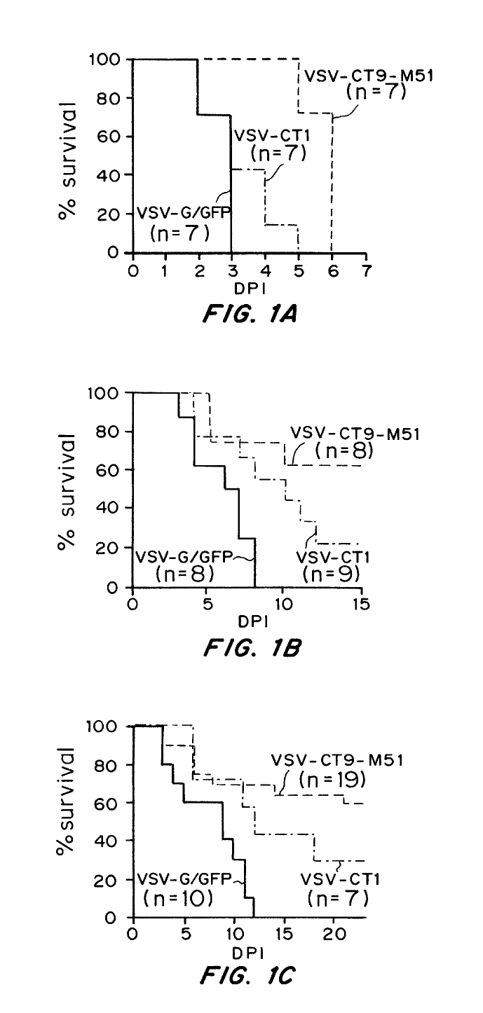

Prior to immunization experiments, the relative toxicity of VSVs within the brain was determined. Introduction of VSV in mice can result in deadly encephalitis and the severity of disease and susceptibility of the animals is far more severe in newborn and immature mice. Sixteen (16)-day-old mice were used to compare the neurovirulence of the two attenuated VSV mutants to that of VSV-G/GFP. Sixteen-day-old Swiss-Webster mice were injected into the forebrain with the VSV strain being tested. A small injection volume of 200 nl was used. VSV-G/GFP, VSV-CT1 and VSV-CT9-M51 injected animals survived an average of 2.7+/−0.5, 3.3+/−1.1 and 5.7+/−0.5 days after intracerebral injections, respectively (FIG. 1A). The survival of VSV-CT9-M51 was significantly longer than for VSV-G/GFP (p<0.05).

### Example 2

### Attenuated VSVs are Less Neuroinvasive than VSV-G/GFP

VSV enters the brain by the olfactory nerve after intranasal inoculation. After reaching the olfactory bulb, VSV can spread to and infect distinct periventricular aminergic nuclei including locus ceruleus and the raphe (van den Pol, et al., J. Virol., 76:1309-1327 (2002)). After an intranasal inoculation of 10,000 PFU of VSV-G/GFP at postnatal day 16 (P16), all animals died at a mean of 5.9+1-2 days. In contrast, 5 out of 8 (62%) of VSV-CT9-M51 treated and 2 out of 9 (22%) of VSV-CT1 inoculated animals were alive and free of neurological complications 28 days after the intranasal dose. In those VSV-CT1 and VSV-CT9-M51 inoculated animals that succumbed to VSV encephalitis the mean duration to death was longer than in VSV-G/GFP treated animals with 8+/−3.2 days and 6.7+/−2.9 days respectively (FIG. 18).

### Example 3

### Reduced Morbidity after Intracerebral Injection of recombinant VSV

Stereotactic intracerebral injections 1,500 PFU of VSV-G/GFP in adult animals resulted in 100% mortality in the injected cohort (n=10). In contrast, partial survival was seen in mice that were injected with the 1,500 PFU of attenuated recombinant VSVs. 11 out of 19 (58%) mice inoculated with VSV-CT9-M51 survived with an initial drop in body weight which recovered within a week (see details below). 2 out of 7 (29%) mice inoculated with VSV-CT1 survived (FIG. 1C). The mean time to morbidity was delayed with the attenuated mutants with 7.4±2.2 days for VSV-CT9-M51, and 9.6±2.2 days for VSV-CT1 compared to 6.7±1.1 days for VSV-G/GFP inoculated mice, though this difference did not reach statistical significance (p=0.56; n=8, n=5, n=10, respectively; ANOVA).

To determine the effect of the attenuated phenotype on the extent and pattern of VSV infection in the brain, serial brain sections were analyzed at 1 and 2 days post inoculation for signs of infection at the injection site and at 5 dpi for assessing viral spread. Both VSV-G/GFP and VSV-CT9-M51 express the green fluorescent reporter gene in individual infected cells around the injection site. The placement of the injection and the extent of infection around the injection site was consistent in all animals regardless of the mutant used. Marked GFP expression was found at the injection site in all 10 animals. Analyzing brains at 3 dpi, a commonly infected brain region was the striatum. Other regions of the brain that showed viral infection included the corpus callosum, septum, cingulate cortex, motor cortex, substantia nigra, and hypothalamus. Infection in the ependyma and periventricular parenchyma was found in the olfactory, lateral and third ventricle in the VSV-G/GFP injected animals. In mice that survived intracranial challenge with VSV-CT9-M51, histological analysis at 3 months post-inoculation showed no residual viral GFP expression. Clear scar formation at the injection track was noted in mice surviving infection.

### Example 4

### Quantitative Comparison of Viral Loads after Intracranial Injection of VSV Variants

Materials and Methods

Quantitative Real-Time PCR

Brain tissues and cell cultures were lysed, and RNA was extracted using TRIzol reagent (Invitrogen) according to the manufacturer's instructions. Primary cultures of adult human glia cells were grown in 12 well plates and infected with VSV-G/GFP or VSV-CT9-M51 at a multiplicity of infection (MOI) of 2 for 6 hours. Triplicate glia cultures infected with each virus and mock-infected cells were lysed and RNA was extracted. Total RNA was reverse transcribed using the SuperScript III RT kit (Invitrogen) with random hexamers. Quantitative PCR was performed using an IQ iCycler Real-Time PCR Thermocycler (Bio-Rad) and the following Taqman Gene Expression assays (ABI): human MxA (Cat No. Hs00182073_ml), human ISG15 (Cat No. Hs00192713_ml), and human GAPDH (Cat No. Hs99999905_ml). The following endogenous control assays (ABI) were also used: human ACTB (Part No. 4333762E), mouse Actb (Part No. 4352933E). A custom Taqman assay with the following specifications was used to detect VSV NIP: Forward primer 5-CCT AAG AGA GAA GAC AAT TGG CAA GT-3′ (SEQ ID NO:1); Reverse primer 5′-TCC ATG ATA TCT GTT AGT TTT TTT CAT ATG TAG CA-3′ (SEQ ID NO:2); FAM dye probe 5′-ACA AAT GAC CCT ATA ATT CT-3′ (SEQ ID NO: 3). All quantitative PCR was performed using the Taqman Gene Expression Master Mix (ABI) and a two-step PCR protocol consisting of a hot start at 95° C. for 10 min, followed by 40 cycles of 95° C. for 15 sec and 60° C. for 1 min. All results were normalized with respect to beta-actin expression.

Results

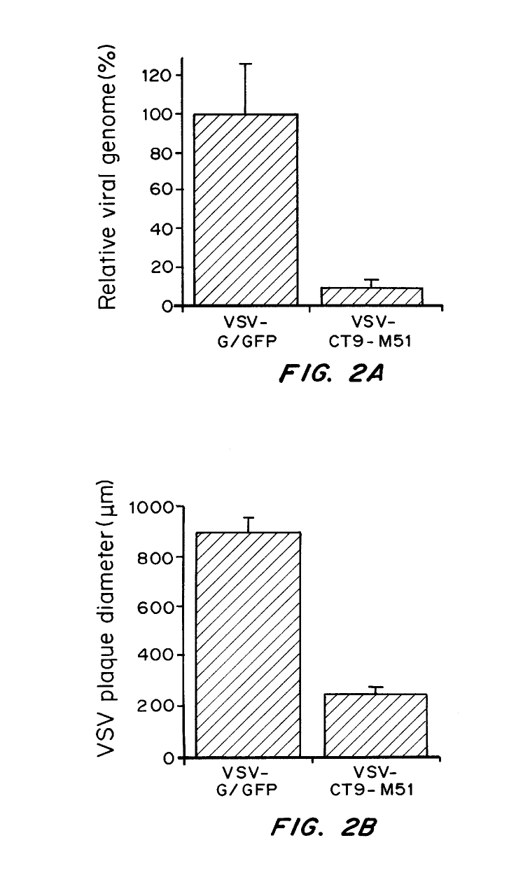

To compare potential differences in replication in the brain, similar viral loads were innoculated intracranially in normal mouse brain (n=8 for VSV-G/GFP; n=7 for VSV-CT9-M51), and the brains were harvested four days later. To determine virus loads, viral genomes were quantified in whole brain using a sequence that spanned the VSV N and P genes using quantitative PCR. The virus with the greatest replication was VSV-G/GFP, and the virus with the least replication was VSV-CT9-M51 (FIG. 2A). Plaque size in vitro were compared for the same two viruses. The plaques and the number of dead cells were considerably larger for VSV-G/GFP than for VSV-CT9-M51 (FIG. 2B).

### Example 5

### No Differences in Spectrum of Cellular Targets after Intracranial Injection Between VSV Variants

Materials and Methods

Immunohistochemistry for Cellular Characterization

After fixation with paraformaldehyde and permeabilization with 0.2% Triton X100 of either infected cell cultures, or mouse brain infected with virus, mouse antisera against the neuronal antigen Neu N and against the astrocyte marker glial fibrillary acidic protein (GFAP), both from Chemicon, were used at a 1:1000 dilution for several hours. After multiple washes, a secondary antibody of goat or donkey anti-mouse antibody conjugated to the red fluorophone Alexa 594(Invitrogen-Molecular Probes, Eugene, Oreg.) was used to immunostain the antigens of interest.

Results

Cellular targets of VSV-G/GFP and VSV-CT9-M51 were investigated both in vivo and in vitro. VSV-G/GFP and VSV-CT9-M51 were used to infect mouse brains (the mice were euthanized two days later) or mouse brain cultures. No detectable difference in the identity of infected cells was found by immunostraining using antisera against Neu N to detect neurons, or GFAP to detect astrocytes of comparable regions infected by the two viruses. Both neurons and astrocytes were infected by each virus.

### Example 6

### Intranasal and Intramuscular Immunization Eliminates VSV Related Morbidity

VSV induces a very strong adaptive-immune response that effectively limits peripheral systemic VSV infections. However, little is known about the extent to which a previous peripheral exposure of VSV may impact its neuropathology. Since intracerebral injection of wildtype-based VSV-G/GFP resulted in fatal encephalitis and attenuated variants showed partial mortality, the ability of immunization to protect mice against an intracerebral challenge of VSV was tested. Five week old Swiss-Webster mice were divided into one non-immunized control group and one group that was immunized either intranasally or with an intranasal/intramuscular combination of VSV-G/GFP. Immunized animals received a secondary immunization of VSV after 6 weeks. Two weeks after the secondary immunization, both immunized and non-immunized mice were challenged with an intracerebral injection of either VSV-G/GFP (n=17; 7 immunized and 10 non-immunized), VSV-CT1 (n=14; 7 immunized and 7 non-immunized) or VSV-CT9-M51 (n=32; 13 immunized and 19 non-immunized).

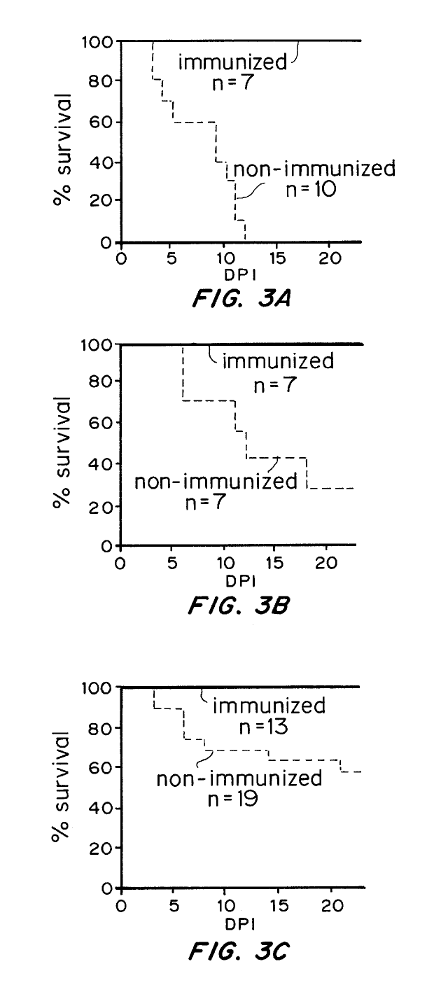

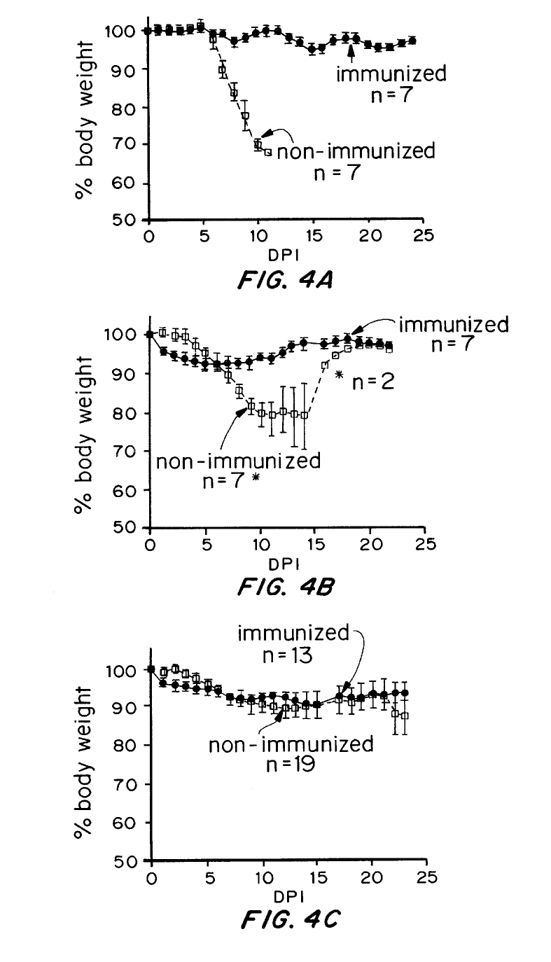

A primary result was that all immunized mice (n=27) survived intracerebral injection of VSV. Even mice receiving VSV-G/GFP, a wildtype-based VSV with a GFP reporter, were completely protected when immunized, whereas all non-immunized control mice receiving intracerebral VSV-G/GFP succumbed to VSV encephalitis (FIGS. 3A, 3B, and 3C) after a mean time to lethality of 6.7±1.1 days (n=10). After intracerebral inoculation, immunized mice did not display signs of distress, the fur remained groomed, and any weight change was moderate (FIGS. 4A, 4B, and 4C). In contrast, many of the non-immunized mice showed severe responses to intracranial VSV inoculation (FIGS. 4A, 4B, and 4C).

Although not quantified, other signs of viral infection were observed including decreases in motor activity and fur changes that were not observed in the immunized animals after intracerebral injections. None of the immunized and VSV-challenged animals, (VSV-G/GFP (n=7), VSV-CT1 (n=7) and VSV-CT9-M51 (n=13)) developed neurological findings during the 3 month observation period following intracranial injection of the virus. Considering the above reported neurological morbidity incidence in VSV-CT1 injected and in VSV-CT9-M51 injected non-immunized animals, respectively, the immunization resulted in a significantly reduced incidence (v0.005, chi square test) of neurological dysfunction.

After intracerebral injection, immunized and non-immunized mice showed weight loss (FIGS. 4A, 4B, and 4C). The maximum weight loss for immunized animals compared to non-immunized control mice was 5.7±1.1 g to 30.4±1.3 g for VSV-G/GFP, 7.0±0.7 g to 21.9±4.6 g for VSV-CT1, and 9.1±1.7 g to 13.5±5.3 g for VSV-CT9-M51.

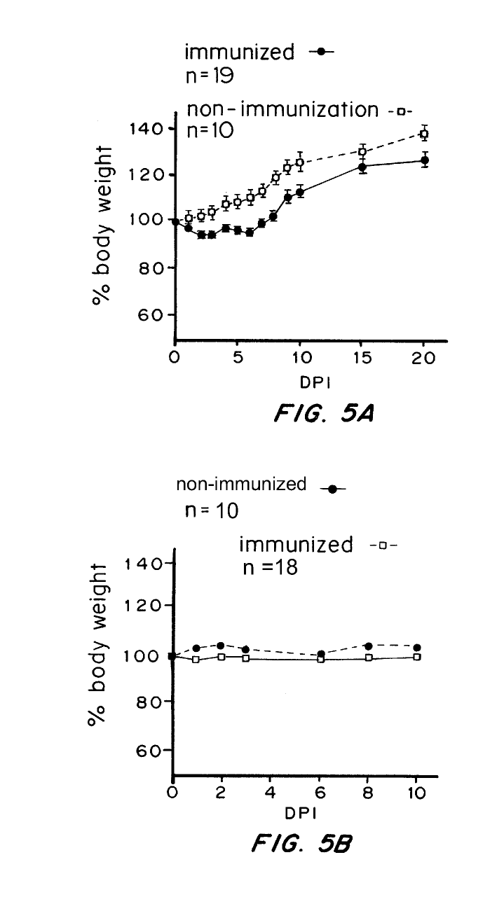

Two routes of immunization were used, intranasal or intramuscular. No difference in animal weight or protective potential was observed between the two paths of immunization, and therefore both groups were pooled. Following primary immunization, mice went through a phase of moderate weight loss compared to non-immunized controls. The weight loss had a rapid onset starting 1 day after virus inoculation and reaching a maximum weight loss of 5.3±0.6% at the second day. Mice started to steadily gain weight after 5 days (FIGS. 5A and 5B). This temporary weight loss is consistent with previous reports using VSV as a vaccination vector (Publicover, et al., J. Virol. 80:7028-36 (2006), Roberts, et al., J. Virol. 73:3723-3732 (1999)). In contrast, the secondary immunization (boost) 6 weeks later did not result in a significant weight loss, reflecting systemic protection through the primary immunization (FIGS. 5A and 5B).

### Example 7

### VSV-CT9-M51 Retains Oncolytic Activity Against a Wide Variety of Cancers In Vitro

Materials and Methods

Cell Culture

U87MG human high grade glioma cells and 4T1 mouse mammary carcinoma cells were obtained from ATCC (Rockville, Md.). Generation of the rU87 cell line stably transfected with the monomeric red fluorescent protein (pDsRed-monomer-C1 (Invitrogen)) was described elsewhere (Wollmann, et al., J. Virol., 81:1479-1491 (2007)). U118MG, U373, A172 human glioma cell lines and a 9 L rat gliosarcoma cell line were kindly provided by Dr. R. Matthews (Yale University). A549, Calu-1 human lung carcinoma and T-47D, MCF-7human breast carcinoma cells were supplied by the Yale Cancer Center (Yale University). Primary cultures of adult human brain cells were prepared from human temporal lobectomy material, removed for intractable epilepsy, solely for the benefit of the patient and the use was approved by the Yale University Human Investigation Committee. In detail, surgery samples were cut into small cubes, digested with 0.25% Trypsin for 15 mins, and placed onto Millipore cell culture filter insets over a medium supply of minimal essential medium (MEM) (Gibco) containing 25% fetal bovine serum (FBS). 5-7 days later, cells that grew onto the filter membrane from the tissue block were harvested and maintained in MEM supplemented with 10% FBS. Astrocytic lineage was confirmed with immunohistochemistry. The following mouse glioma cell lines were used in the study: CT2A cells were a gift from Dr. T. Seyfried (Boston College-Chestnut Hill, Mass.), DBT mouse glioma cells were kindly supplied by Dr. J. O. Fleming (University of Wisconsin-Madison, Wis.). Cells were maintained in MEM (Gibco) supplemented with 10% FBS, 1% sodium pyruvate, 100 uM non-essential amino acids, 10 mM HEPES buffer.

Results

The experiments above demonstrate that VSV-CT9-M51 shows the most attenuated neurovirulence of the VSVs compared here, and that immunization provides further protection against VSV-CT9-M51 injections into the brain. VSV has been proposed as a potential oncolytic virus that can target various forms of cancer (Stojdl, et al., Cancer Cell, 4:263-275 (2003)). Modifications in the VSV genome that generate attenuated phenotypes may alter their oncolytic potency. To test whether VSV-CT9-M51 retained its oncolytic activity against a wide variety of human cancers, 9 different human cancer cell lines were tested, which included 4 different high grade gliomas (A172, U87, U118, U373), 2 lung cancer (calu-1, A-549) and 2 breast cancer cells (T47-D, MCF-7). Breast and lung cancer cell lines were included in this battery since systemic cancers can metastasize to the brain and are in fact more commonly observed than primary brain malignancies. All 4 glioma cell lines tested were susceptible to complete VSV mediated cell destruction. An MOI of 1 led to >95% cell death at 24 hpi for U118, 36 hpi for U87, 48 hpi for A-172, and 72 hpi for U373 cells. The two lung cancer lines showed >95% cell death at 24 hpi (A-549) and 120 hpi (calu-1), respectively. The two breast cancer lines showed >95% cell death at 36 hpi (MCF-7) and 72 hpi (T47-D), respectively.

Due to the possibility of utilizing syngeneic rodent cancer models VSV-CT9-M51 was also tested on mouse and rat cancer cell lines. After infecting with an MOI of 1 with VSV-CT9-M51, >95%+ cell death was observed at 24 hpi for rat 9 L gliosarcoma cultures, 36 hpi for DBT mouse glioblastoma cells, and 48 hpi for CT2A mouse glioma. Together, these data show that despite its attenuated phenotype VSV-CT9-M51 remains highly oncolytic against a multitude of both human and rodent cancer cell lines.

### Example 8

### Human Brain Cells Mount a Robust Interferon Response to VSV-CT9-M51

Materials and Methods

Immunofluorescence for IRF3 and OAS

For interferon regulatory factor 3 (IRF3) and cleaved caspase 3 immunocytochemistry adult human astrocytes on 6 mm spot slides (Electron Microscopy Sciences, Fort Washington, Pa.) were fixed in 4% paraformaldehyde and incubated with a 1:100 dilution of polyclonal rabbit anti-IRF3 antiserum (Santa Cruz Biotechnology, Santa Cruz, Calif.) or a 1:100 dilution of rabbit anti-cleaved caspase antiserum (Cell Signaling-MA), followed by detection with a 1:200 dilution of donkey anti-rabbit immunoglobulin—Alexa Fluor 594.

Results

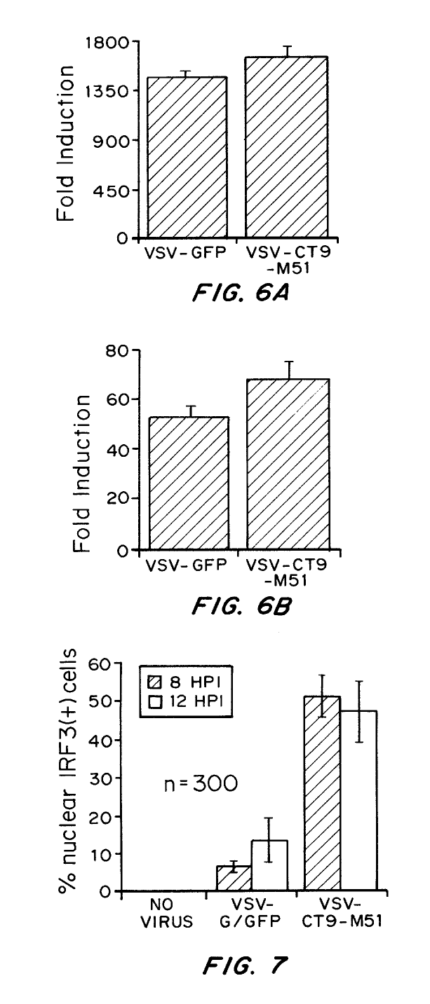

One mechanism that may account for the reduced neurovirulence of VSV-CT9-M51 is a reduced ability of the virus to replicate, and to block the intrinisie interferon-inducible gene response of infected cells. The M protein of VSV blocks transport of cellular mRNA's, including the antiviral protein interferon. VSV mutants with an M51 genotype are impaired in their capability to interfere with cellular mRNA transport (Stojdl, et al., Cancer Cell, 4:263-275 (2003)), and therefore may induce a stronger cellular immune response (Ahmed, et al., J. Virol., 82:9273-9277 (2008)). As VSV-CT9-M51 showed reduced neurovirulence compared with VSV-CT1, focus was drawn to VSV-CT9-M51 and its parent virus, VSV-G/GFP. The response of adult human astrocytes to VSV-G/GFP and VSV-CT9-M51 was compared using quantitative RT-PCR. Primary cultures were established from human temporal lobectomy material and immunostaining for GFAP revealed >95% astrocyte origin. Six hours post infection (HPI) a statistically significant increase was observed in expression of two interferon-induced genes in all VSV treated cultures (FIGS. 6A and 6B) (p<0.05; ANOVA). To support these findings immunostaining was carried out for the transcription factor IRF-3, which translocates from the cytoplasm to the nucleus during an innate cellular immune response. A greater number of cells showed a nuclear location of IRF-3 in response to VSV-CT9-M51 than to VSV-GFP, and the difference was highly significant at 8 and 12 HPI (p<0.05, t-test) with an MOI of 10 (FIG. 7). Together these data suggest that VSV-CT9-M51 may be less invasive in the brain due to the reduced ability of the virus to block intrinsic antiviral defenses. Immunostaining against GFAP was used to confirm the astrocytic nature of the primary cultures used for these experiments.

The crucial role of intrinsic immunity is illustrated by mice lacking IFN-receptors that show enhanced mortality upon VSV infection despite having an intact systemic immune system (Muller, Science, 264:1918-1921 (1994)). Interferon inducible-genes play an essential role in defending the brain from VSV infection (Trottier, et al., Virology, 333:215-225 (2005), Wollmann, et al., J. Virol., 81:1479-1491 (2007)). In all three experiments comparing the neurovirulence of the three VSVs used here, VSV-CT9-M51 was the least neurovirulent. It is believed that this is in part due to a shift in the ability of the intrinsic immune system to block viral infections. It was discovered that IFN-induced downstream genes ISG15 and MxA were induced to a greater extent in control adult human astrocytes by VSV-CT9-M51 compared with the other tested strains of VSV. Similarly, VSV-CT9-M51 induced more nuclear translocation of IRF3 than did VSV-G/GFP. Primary cultures from human epileptic foci were used as controls for human tumor cells. Although possibly not normal, these cells are non-malignant and non-transformed in nature, and hence may serve as controls for cancer cells. Finally, VSV-CT9-M51 showed reduced replication in vivo, and reduced plaque size and cell death on normal brain cells in vitro compared with VSV-G/GFP.

### Example 9

### Systemic VSV-CT9-M51 Targets Brain Tumors

In previous studies with a different virus, VSVrp30 proved capable of targeting and spreading within brain tumors and peripheral glioma grafts and killing tumor cells (Ozduman, et al., J. Neurosci. 28:1882-1893 (2008)). As described above, VSV-CT9-M51 had an attenuated virulence in the brain and was able to efficiently kill glioma cells in vitro. To determine if this attenuated VSV strain would still be able to target glioblastoma in an in vivo intracranial brain tumor model, as a proof of principle, human glioblastoma (U87) cells that expressed a red reporter gene were transplanted into the striatum of SCID mice deficient in B and T cells that had not been immunized. Tumors grew with no signs of immune-mediated rejection.

Virus was given in the tail vein. VSV-CT9-M51 targeted, infected, spread within, and killed brain tumor xenografts. When mice with cerebral tumors at 72 hours post inoculation, were analyzed by immunostaining of tissue sections, selective VSV-CT9-M51 infection was found in all 6 (100%) of striatal rU87 glioma xenografts in 3 animals. Apoptotic cell death in the infected tumor cells was documented using immunohistochemistry with an antibody recognizing cleaved caspase 3, a late effector of apoptosis. A mean of 97.9%+/−2.6 of the cells stained positively for the antibody. Multifocal tumors within the brain were simulateously infected after intravenous injections, and the infection within the tumor was highly specific for the tumor cells. Single red fluorescent tumor cells outside the main tumor bulk and infiltrating the normal brain parenchyma were found infected with VSV-CT9-M51, without infection of the surrounding normal brain parenchyma.

Control experiments were conducted to test if VSV-CT9-M51 entered the normal brain (not bearing a tumor) upon systemic injection of the virus. Seventy-two hours after intravenous injection of 10⁷ PFU in SCID mice, no infected cells were found in consecutive serial brain sections, indicating the virus selectively infected the tumor.

Together, these data show that VSV-CT9-M51 even with two attenuating mutations involving M51 and a reduction in the length of the cytoplasmic tail of the G protein, is still able to target selectively, spread within, and kill human glioblastoma after intravenous inoculation. This dual recombinant VSV not only can target brain tumors, but also shows reduced intrinsic neurovirulence upon injection into the brain and after intranasal application, and can be further controlled by peripheral immunization.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A method for decreasing toxicity to non-target tissue during therapy of an individual with a therapeutic virus comprising immunizing the individual to the therapeutic virus prior to administration of the therapeutic virus.

2. The method of claim 1 wherein the therapeutic virus is an oncolytic virus and the individual has cancer.

3. The method of claim 1 wherein the individual is immunized by administration of an effective amount of an immunizing virus selected from the group consisting of infectious virus, viral subunits, viral proteins and antigenic fragments thereof, nucleic acids encoding viral subunits, antigenic proteins or polypeptides.

4. The method of claim 1 wherein the target tissue is brain tumor and the immunizing virus is administered by intranasal or intramuscular injection.

5. The method of claim 1 wherein the immunizing virus is administered more than once before administration of the therapeutic virus.

6. The method of claim 1 wherein the immunizing and therapeutic viruses are VSV viruses.

7. The method of claim 1 wherein the immunizing virus is an attenuated therapeutic virus or a different strain of the therapeutic virus.

8. The method of claim 1 further comprising immunosupressing the individual during the time of initial treatment with the therapeutic virus.

9. The method of claim 1 wherein the therapeutic virus is selected from the group consisting of VSV-CT1, VSV-CT9, and VSV-CT9-M51.

10. The method of claim 1 wherein the therapeutic virus is engineered to express one or more additional genes encoding proteins selected from the group consisting of targeting proteins, antigenic proteins, and therapeutic proteins.

11. The method of claim 2 wherein the tumor is a brain tumor selected from the group consisting of glioblastomas, oligodendrogliomas, meningiomas, supratentorial ependymonas, pineal region tumors, medulloblastomas, cerebellar astrocytomas, infratentorial ependymonas, brainstem gliomas, schwannomas, pituitary tumors, craniopharyngiomas, optic gliomas, and astrocytomas.

12. The method of claim 1 wherein the therapeutic virus is genetically engineered to produce a targeting protein, therapeutic protein, or prophylactic protein.

13. The method of claim 1 wherein the therapeutic virus is a vesicular stomatitis oncolytic virus comprising a truncation of the cytoplasmic tail of the G protein and a deletion of one or more amino acids in the M protein.

14. The method of claim 13 wherein the virus comprises a truncation of the cytoplasmic tail of the G protein and a deletion of one or more amino acids in the M protein.

15. An oncolytic virus composition comprising an effective amount of a vesicular stomatitis oncolytic virus comprising a truncation of the cytoplasmic tail of the G protein and a deletion of one or more amino acids in the M protein.

16. The virus of claim 15 comprising a truncation of the cytoplasmic tail of the G protein to 9 amino acids and a deletion of the fifty-first (51) amino acid of the M protein.

17. A kit comprising an immunizing virus and a therapeutic virus.

18. The kit of claim 17 wherein the therapeutic virus is an oncolytic virus.

19. The kit of claim 17 for use in the method of claim 1.
